(19) United States
(12) Patent Application Publication
(10) Pub. No.: US 2025/0332159 A1
(43) Pub. Date: Oct. 30, 2025

(54) REACTIVE OXYGEN SPECIES MODULATION THROUGH BINDING OF LIGANDS AT THE NQ-BINDING SITE OF THE RESPIRATORY COMPLEX III

(71) Applicant: University of Health Sciences & Pharmacy in St. Louis, St. Louis, MO (US)

(72) Inventor: Muhammad M. HAGRAS, St. Louis, MO (US)

(73) Assignee: University of Health Sciences & Pharmacy in St. Louis, St. Louis, MO (US)

(21) Appl. No.: 19/189,936

(22) Filed: Apr. 25, 2025

Related U.S. Application Data
(60) Provisional application No. 63/638,833, filed on Apr. 25, 2024.

(51) Int. Cl. A61K 31/495; A61K 31/136; A61K 31/167; A61K 31/40; A61K 31/417; A61K 31/4196; A61K 31/437; A61K 31/4409; A61K 31/47; A61K 31/4985; A61K 31/506; A61K 31/55

(52) U.S. Cl. CPC A61K 31/495; A61K 31/136; A61K 31/167; A61K 31/40; A61K 31/417; A61K 31/4196; A61K 31/437; A61K 31/4409; A61K 31/47; A61K 31/4985; A61K 31/506; A61K 31/55

(57) ABSTRACT

In some aspects, the present disclosure provides methods for modulating the level of reactive oxygen species. In some embodiments, the present methods involve the use of compounds that bind to respiratory complex III at the NQ-binding site. The present methods may be used for treatment or prevention of a disease or disorder, for example a disease or disorder characterized by or associated with dysregulated levels of reactive oxygen species.

This application claims the benefit of and the priority to U.S. Provisional Patent Application 63/638,833, filed on Apr. 25, 2024, which is hereby incorporated by reference in its entirety for all purposes.

### BACKGROUND

### 1. Field

The present disclosure relates generally to the field of pharmaceuticals and active pharmaceutical ingredients. In particular, the compounds described herein may be used as therapeutic agents for multiple diseases.

### 2. Description of Related Art

Through controlled regulation of the cellular and mitochondrial ROS levels, multiple disorders such as cancer, musculoskeletal disorders such as rheumatoid arthritis and osteoarthritis, and neurodegenerative diseases such as Alzheimer's and Parkinson's diseases can be studied, where ROS plays a pivotal role in their underlying pathogenesis. In particular, these studies would enhance understanding of the role of ROS in the progression of those disorders, rendering ROS a potential therapeutic target.

Reactive oxygen species (ROS) are by-products of normal cellular aerobic metabolism, which includes various compounds such as superoxide radical anion (O^{•-}), hydroxyl radical (OH·^{•}), and hydrogen peroxide (H₂O₂). ROS play a crucial role in several cellular processes, including cellular signaling pathways and maintaining the immune system, and are implicated in various essential physiological functions such as cell cycle progression and proliferation (Boonstra, 2004; Yang et al., 2013; Zhang et al., 2016). Therefore, imbalances in ROS contribute to the development and progression of multiple diseases such as cancer (Waris and Ahsan, 2006), musculoskeletal disorders such as rheumatoid arthritis and osteoarthritis (López-Armada et al., 2013; Bolduc et al., 2019; Abbas and Monireh, 2008; McGarry et al., 2018), and neurodegenerative diseases such as Alzheimer's and Parkinson's diseases (Source and Krouse, 2009; Manoharan et al., 2016).

ROS are generated naturally by multiple enzymes, including respiratory complexes such as respiratory complex III (a.k.a. bc₁ complex). Electrons that pass through the bc₁ complex are a primary source of mitochondrial ROS, specifically the superoxide radical ion (O₂^{•-}) that is produced during the oxidative phosphorylation process (Drose and Brandt, 2008; Lanciano et al., 2013). Disruptions in the bc₁ complex can result in ROS imbalances, and therefore, strategies for modulating the activity of the bc₁ complex could have therapeutic implications in an array of human diseases, including multiple forms of cancer, such as breast cancer. Interestingly, ROS was recently recognized as a “double-edged sword” and is found to be the underlying mechanism for most of the anticancer therapeutic methods through elevating the cellular levels of ROS above the apoptotic threshold level, thereby triggering apoptosis in cancer cells while leaving the normal cells at the under-the-threshold level of ROS (Yang et al., 2016; Watson, 2013; Trachootham et al., 2009).

The bc₁ complex is a homodimer where each monomer encompasses four redox centers (Fe₂S₂, heme b_{L}, heme b_{H}, and heme c₁) and two native binding sites (Qₒ and Qᵢ sites). Electrons flow in the bc₁ complex in a series of protonmotive ET reactions known as Q-cycle, proposed by Mitchell (Mitchell, 1961; Mitchell, 1975). Upon binding the ubiquinol (UQH₂) molecule at the Qₒ site, one electron of the bound UQH₂ molecule transfers to the [2Fe-2S] cluster of the Rieske domain, docked at the proximal docking site. Another electron transfers to heme b_{L}, which subsequently passes it to heme b_{H}, and finally to a bound ubiquinone (UQ) or semiquinone (SQ) molecule bound at the Qᵢ-site (Mitchell, 1976; Brandt and Trumpower, 1994; Crofts et al., 1999; Crofts et al., 2003; Osyczka et al., 1999). Rieske domain undergoes a domain movement of ˜22 Å to bind at the distal docking site, where [2Fe-2S] cluster passes its electron to heme c₁, which in turn passes it to heme c of the water-soluble cytochrome c carrier (Crofts et al., 1999; Zhang et al., 1998). The enzyme turnover takes two Q-cycles to collectively transport 4 protons to the membrane's positive side, uptake 2 protons from the negative side, reduce two cytochrome c molecules, oxidize two ubiquinol molecules, and reduce one ubiquinone molecule (FIG. 1) (Brandt and Trumpower, 1994; Crofts et al., 2003; Osycka et al., 2005).

The atomistic details of the tunneling pathways and the corresponding ET rates between all redox pairs in the bc₁ complex have been calculated. Interestingly, it was discovered that the electron transfer between the heme b_{L} and the heme b_{H} redox centers is controlled by a key phenylalanine residue (Phe90) that primarily can assume two different conformations (a.k.a. ON/OFF conformations) (Hagras, 2024; Hagras et al., 2015; Hagras and Stuchebrukhov, 2021; Hagras and Stuchebrukhov, 2016). The Phe90 residue only exists in the ON conformation when the Qₒ-site is occupied. Additionally, extensive MD simulations were performed that confirmed previous discoveries regarding the role of Phe90 residue as an ET switch or an ET gate, whose conformation influences the rate of the ET reaction between heme b_{L} and heme b_{H} redox pairs significantly. A novel orphan binding site (NQ-site) in the bc₁ complex that has never been characterized before was discovered (FIG. 2) (Hagras and Stuchebrukhov, 2021; Hagras and Stuchebrukhov, 2016). The NQ-binding site is deep enough to modulate the Phe90 conformation and thus modulate the ET between heme b_{L} and heme b_{H} redox centers.

There remains a need to develop new compounds that modulate reactive oxygen species production, which will aid in the understanding of multiple disorders and develop novel therapeutics targeting those diseases.

### SUMMARY

In some aspects, the present disclosure provides compounds which bind preferentially at the NQ-binding site of respiratory complex III. In some embodiments, the present methods facilitate the modulation of levels of reactive oxygen species (ROS). In some embodiments, the present methods facilitate an elevation in ROS levels. In some embodiments, the present methods facilitate a reduction in ROS levels. In some embodiments, the present methods may be useful for elevating ROS levels in a breast cancer cell. In some embodiments, the present methods may be useful for lowering ROS levels in a breast cancer cell. In some embodiments, the present methods may be useful for raising ROS levels in a healthy cell. In some embodiments, the present methods may be useful for lowering ROS levels in a healthy cell. In some embodiments, the present methods may be useful for raising ROS levels in a cell that is not healthy, such as a cancer cell. In some embodiments, the present methods may be useful for treating a viral infection. In some embodiments, the present methods may be useful for treating a bacterial infection. In some embodiments, the present methods may be useful for treating a fungal infection. In some embodiments, the present methods may be useful for lowering ROS levels in a cell that is not healthy, such as a cancer cell.

In some embodiments, the present methods may facilitate modulating the ROS levels, and as such be useful for antibiotic or anticancer purposes. In some embodiments, the present methods may facilitate lowering of ROS levels, and as such be beneficial for the treatment or prevention of diseases or disorders that are treatable or preventable with an anti-inflammatory agent or neuroprotective agent or anti-inflammatory agents.

In some embodiments, the present methods may facilitate an elevation of ROS levels, and as such be useful for antibiotic, or anticancer purposes. In some embodiments, the present methods may facilitate an elevation of ROS levels, and as such beneficial for the treatment or prevention of diseases or disorders that are treatable or preventable with an anticancer agent or an antibiotic agent.

In some embodiments, the compound increases the reactive oxygen species in an unhealthy cell. In some embodiments, the compound does not increase the reactive oxygen species in a healthy cell. In some embodiments, the compound increases the reactive oxygen species in an unhealthy cell, but not in a healthy cell. As used herein, the term “healthy cell” means one or more cells or cells of an organism that shows no physiological sign of a disease or disorder. The disease or disorder may be one associated with chronic inflammation. Some non-limiting examples

In some aspects, the present disclosure provides methods of modulating the production of a reactive oxygen species comprising contacting a cell with a compound in an amount sufficient to induce a change in the amount of one or more reactive oxygen species, wherein:
(A) the compound comprises a spirocyclic group;
(B) the compound comprises one or more heteroaromatic groups that contains at least two nitrogen heteroatoms;
(C) the compound comprises one or more sulfonamide groups;
(D) the compound comprises two or more lactams;
(E) the compound comprises one or more heterocycloalkane groups that contain a nitrogen atom in combination with an oxygen atom or a sulfur atom;
(F) the compound comprises one or more piperazine ring;
(G) the compound comprises a fused hetetocycloalkane ring system containing at least two or more ring system one or more nitrogen atoms with at least one seven membered ring;
(H) the compound comprises two or more pendant aromatic ring systems, wherein at least one of the aromatic ring system is a heteroaromatic ring system with at least one nitrogen atom;
(I) the compound comprises two or more fused cycoalkane ring systems, wherein at least one of the cycloalkane rings is a cyclopentane ring, wherein the cycloalkane rings optionally contain one or more heteroatoms;
(J) the compound comprises a triazole group;
(K) the compound is further defined by the formula:

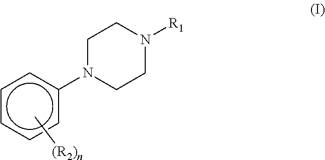

wherein:
   R₁ is C1-C12 alkyl or C1-C12 substituted alkyl; and
   Each R₂ are each independently selected from amino, halo, hydroxy, C1-C6 alkylamino, substituted C1-C6 alkylamino, C2-C12 dialkylamino, or substituted C2-C12 dialkylamino, or
further defined by the formula:

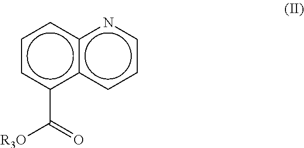

wherein:
   R₃ is hydrogen, C1-C8 alkyl or C1-C8 substituted alkyl; or
further defined by the formula:

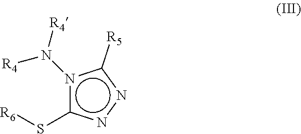

wherein:
   R₄, R₄′, and R₅ are each independently selected from hydrogen, C1-C8 alkyl or C1-C8 substituted alkyl; and
   R₆ is C7-C12 alkyl or C7-C12 substituted alkyl; or
further defined by the formula:

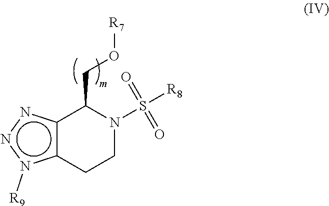

wherein:
   m is 1, 2, 3, or 4;
   R₇ is C7-C12 alkyl or C7-C12 substituted alkyl; and
   R₈ and R₉ are each independently selected from hydrogen, C1-C8 alkyl or C1-C8 substituted alkyl; or
further defined by the formula:

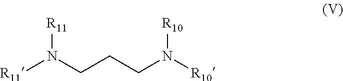

wherein:
   R₁₀, R₁₀′, R₁₀, and R₁₀′ are each independently hydrogen, C1-C8 alkyl, C1-C8 substituted alkyl, C6-C12 aryl or C6-C12 substituted aryl; or
further defined by the formula:

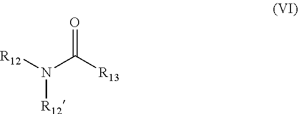

wherein:
   R₁₂, R₁₂′, and R₁₃ are each independently hydrogen, C1-C8 alkyl, C1-C8 substituted alkyl, C6-C12 aryl or C6-C12 substituted aryl; or
further defined by the formula:

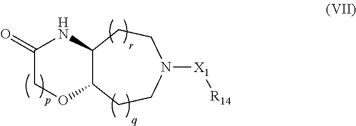

wherein:
   p, q, and r are each independently 0, 1, 2, or 3;
   X₁ is C1-C6 alkanediyl or C1-C6 substituted alkanediyl; and
   R₁₄ is C6-C12 aryl, C6-C12 substituted aryl, C1-C12 heteroaryl or C1-C12 substituted heteroaryl; or
further defined by the formula:

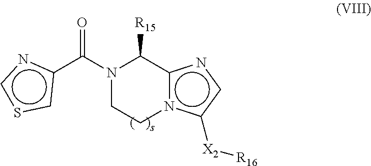

wherein:
   s is 0, 1, 2, or 3;
   X₂ are C1-C6 alkanediyl or C1-C6 substituted alkanediyl;
   R₁₅ is C1-C8 alkyl or C1-C8 substituted alkyl; and
   R₁₆ is amino, hydroxy, C1-C12 alkoxy, C1-C12 substituted alkoxy, C1-C12 alkylamino, C1-C12 substituted alkylamino C1-C12 dialkylamino or C1-C12 substituted dialkylamino; or
further defined by the formula:

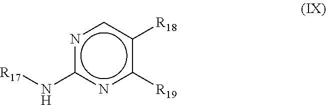

wherein:
   R₁₇, R₁₈, and R₁₉ are each independently C1-C12 heteroaryl or C1-C12 substituted heteroaryl; or
further defined by the formula:

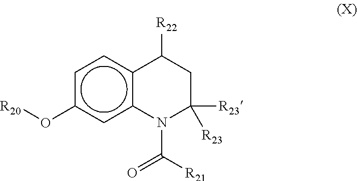

wherein:
   R₂₀, R₂₁, R₂₂, R₂₃, and R₂₃′ are each independently C1-C8 alkyl or C1-C8 substituted alkyl; or
further defined by the formula:

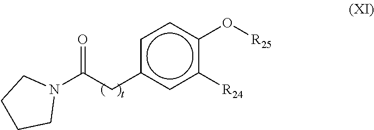

wherein:
   t is 1, 2, or 3; and
   R₂₄ and R₂₅ are each independently C1-C8 alkyl or C1-C8 substituted alkyl; or
further defined by the formula:

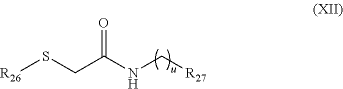

wherein:
   u is 1, 2, 3, 4, or 5; and
   R₂₆ and R₂₇ are each independently C6-C12 aryl, C6-C12 substituted aryl, C1-C12 heteroaryl or C1-C12 substituted heteroaryl; or
further defined by the formula:

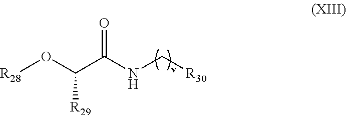

wherein:
   v is 1, 2, 3, 4, or 5; and
   R₂₈, R₂₉, and R₃₀ are each independently C1-C12 alkyl, C1-C12 substituted alkyl, C6-C12 aryl, C6-C12 substituted aryl, C1-C12 heteroaryl or C1-C12 substituted heteroaryl; and/or
(L) the compound is defined as shown in Table 1.

In some embodiments, the compound is further defined by two or more elements defined in (A)-(K) as described above. In some embodiments, the compound is further defined by three or more elements defined in (A)-(K) as described above. In some embodiments, the compound is further defined by the formula:

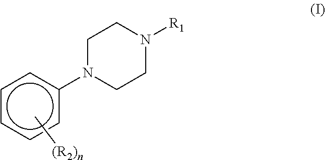

wherein:
R₁ is C1-C12 alkyl or C1-C12 substituted alkyl; and
each R₂ are each independently selected from amino, halo, hydroxy, C1-C6 alkylamino, substituted C1-C6 alkylamino, C2-C12 dialkylamino, or substituted C2-C12 dialkylamino.

In other embodiments, the compound is further defined by the formula:

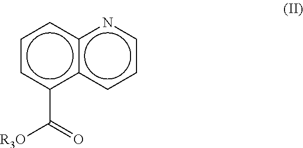

wherein:
R₃ is hydrogen, C1-C8 alkyl or C1-C8 substituted alkyl.

In other embodiments, the compound is further defined by the formula:

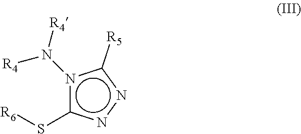

wherein:
R₄, R₄′, and R₅ are each independently selected from hydrogen, C1-C8 alkyl or C1-C8 substituted alkyl; and
R₆ is C7-C12 alkyl or C7-C12 substituted alkyl.

In other embodiments, the compound is further defined by the formula:

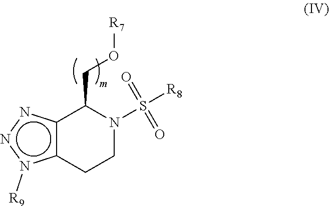

wherein:
m is 1, 2, 3, or 4;
R₇ is C7-C12 alkyl or C7-C12 substituted alkyl; and
R₈ and R₉ are each independently selected from hydrogen, C1-C8 alkyl or C1-C8 substituted alkyl.

In other embodiments, the compound is further defined by the formula:

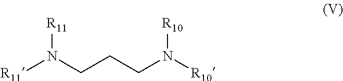

wherein:
R₁₀, R₁₀′, R₁₀, and R₁₀′ are each independently hydrogen, C1-C8 alkyl, C1-C8 substituted alkyl, C6-C12 aryl or C6-C12 substituted aryl.

In other embodiments, the compound is further defined by the formula:

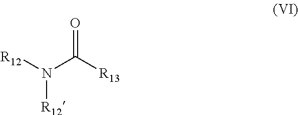

wherein:
R₁₂, R₁₂′, and R₁₃ are each independently hydrogen, C1-C8 alkyl, C1-C8 substituted alkyl, C6-C12 aryl or C6-C12 substituted aryl.

In other embodiments, the compound is further defined by the formula:

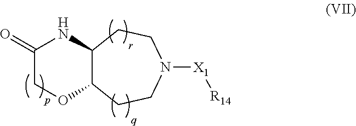

wherein:
p, q, and r are each independently 0, 1, 2, or 3;
X₁ is C1-C6 alkanediyl or C1-C6 substituted alkanediyl; and
R₁₄ is C6-C12 aryl, C6-C12 substituted aryl, C1-C12 heteroaryl or C1-C12 substituted heteroaryl.

In other embodiments, the compound is further defined by the formula:

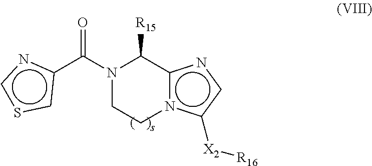

wherein:
s is 0, 1, 2, or 3;
X₂ are C1-C6 alkanediyl or C1-C6 substituted alkanediyl;
R₁₅ is C1-C8 alkyl or C1-C8 substituted alkyl; and
R₁₆ is amino, hydroxy, C1-C12 alkoxy, C1-C12 substituted alkoxy, C1-C12 alkylamino, C1-C12 substituted alkylamino C1-C12 dialkylamino or C1-C12 substituted dialkylamino.

In other embodiments, the compound is further defined by the formula:

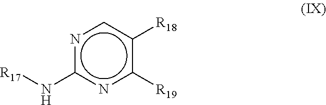

wherein:
R₁₇, R₁₈, and R₁₉ are each independently C1-C12 heteroaryl or C1-C12 substituted heteroaryl.

In other embodiments, the compound is further defined as:

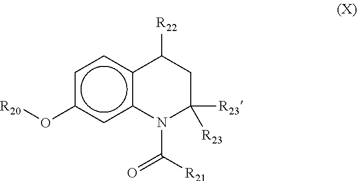

wherein:
R₂₀, R₂₁, R₂₂, R₂₃, and R₂₃′ are each independently C1-C8 alkyl or C1-C8 substituted alkyl.

In other embodiments, the compound is further defined by the formula:

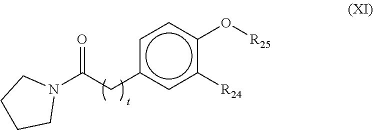

wherein:
t is 1, 2, or 3; and
R₂₄ and R₂₅ are each independently C1-C8 alkyl or C1-C8 substituted alkyl.

In other embodiments, the compound is further defined by the formula:

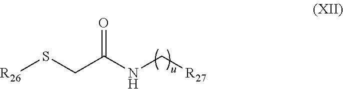

wherein:
u is 1, 2, 3, 4, or 5; and
R₂₆ and R₂₇ are each independently C6-C12 aryl, C6-C12 substituted aryl, C1-C12 heteroaryl or C1-C12 substituted heteroaryl.

In other embodiments, the compound is further defined by the formula:

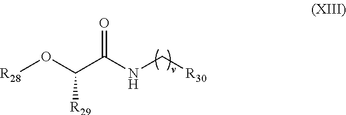

wherein:
v is 1, 2, 3, 4, or 5; and
R₂₈, R₂₉, and R₃₀ are each independently C1-C12 alkyl, C1-C12 substituted alkyl, C6-C12 aryl, C6-C12 substituted aryl, C1-C12 heteroaryl or C1-C12 substituted heteroaryl.

In some embodiments, the compound is further defined by Table 1. In some embodiments, the methods result in decreased production of reactive oxygen species. In other embodiments, the methods result in increased production of reactive oxygen species. In other embodiments, the methods result modulation of one reactive oxygen species. In other embodiments, the methods result modulation of two or more reactive oxygen species.

In some embodiments, the cell is in vitro. In other embodiments, the cell is in vivo. In some embodiments, the cell is in vivo and the method results in the treatment of a disease or disorder. In some embodiments, the disease or disorder is treated by increasing the production of reactive oxygen species. In other embodiments, the disease or disorder is treated by decreasing the production of reactive oxygen species. In some embodiments, the disease or disorder is treated by a combination of pre-treatment with compounds that decrease the production of reactive-oxygen species and then a subsequent treatment with different compounds that increase the production of reactive oxygen species. In other embodiments, the disease or disorder is treated by a combination of pre-treatment with compounds that increase the production of reactive-oxygen species and then a subsequent treatment with different compounds that decrease the production of reactive oxygen species.

In yet another aspect, the present disclosure provides methods of treating a disease or disorder in a patient in need thereof comprising administering to the patient a therapeutically effective amount of a reactive oxygen species modulator, wherein the reactive oxygen modulator is further defined as a compound comprising one or more of the following features:
(A) the compound comprises a spirocyclic group;
(B) the compound comprises one or more heteroaromatic groups that contains at least two nitrogen heteroatoms;
(C) the compound comprises one or more sulfonamide groups;
(D) the compound comprises two or more lactams;
(E) the compound comprises one or more heterocycloalkane groups that contain a nitrogen atom in combination with an oxygen atom or a sulfur atom;
(F) the compound comprises one or more piperazine ring;
(G) the compound comprises a fused hetetocycloalkane ring system containing at least two or more ring system one or more nitrogen atoms with at least one seven membered ring; (H) the compound comprises two or more pendant aromatic ring systems, wherein at least one of the aromatic ring system is a heteroaromatic ring system with at least one nitrogen atom;
(I) the compound comprises two or more fused cycoalkane ring systems, wherein at least one of the cycloalkane rings is a cyclopentane ring, wherein the cycloalkane rings optionally contain one or more heteroatoms;
(J) the compound comprises a triazole group;
(K) the compound is further defined by the formula:

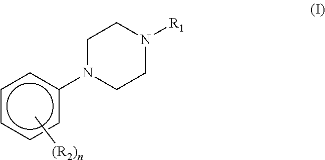

wherein:
   R₁ is C1-C12 alkyl or C1-C12 substituted alkyl; and
   Each R₂ are each independently selected from amino, halo, hydroxy, C1-C6 alkylamino, substituted C1-C6 alkylamino, C2-C12 dialkylamino, or substituted C2-C12 dialkylamino, or
further defined by the formula:

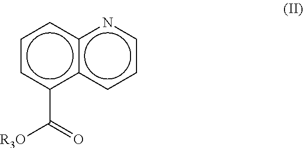

wherein:
   R₃ is hydrogen, C1-C8 alkyl or C1-C8 substituted alkyl; or
further defined by the formula:

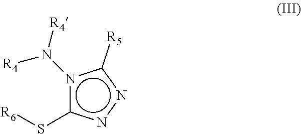

wherein:
   R₄, R₄′, and R₅ are each independently selected from hydrogen, C1-C8 alkyl or C1-C8 substituted alkyl; and
   R₆ is C7-C12 alkyl or C7-C12 substituted alkyl; or
further defined by the formula:

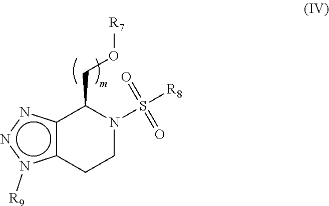

wherein:
   m is 1, 2, 3, or 4;
   R₇ is C7-C12 alkyl or C7-C12 substituted alkyl; and
   R₈ and R₉ are each independently selected from hydrogen, C1-C8 alkyl or C1-C8 substituted alkyl; or
further defined by the formula:

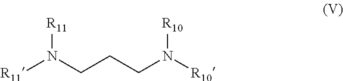

wherein:
   R₁₀, R₁₀′, R₁₀, and R₁₀′ are each independently hydrogen, C1-C8 alkyl, C1-C8 substituted alkyl, C6-C12 aryl or C6-C12 substituted aryl; or
further defined by the formula:

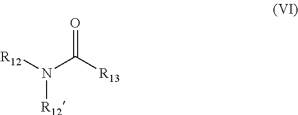

wherein:
   R₁₂, R₁₂′, and R₁₃ are each independently hydrogen, C1-C8 alkyl, C1-C8 substituted alkyl, C6-C12 aryl or C6-C12 substituted aryl; or
further defined by the formula:

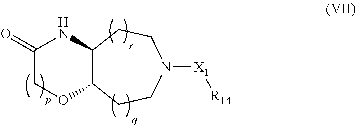

wherein:
   p, q, and r are each independently 0, 1, 2, or 3;
   X₁ is C1-C6 alkanediyl or C1-C6 substituted alkanediyl; and
   R₁₄ is C6-C12 aryl, C6-C12 substituted aryl, C1-C12 heteroaryl or C1-C12 substituted heteroaryl; or
further defined by the formula:

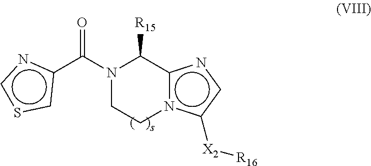

wherein:
   s is 0, 1, 2, or 3;
   X₂ are C1-C6 alkanediyl or C1-C6 substituted alkanediyl;
   R₁₅ is C1-C8 alkyl or C1-C8 substituted alkyl; and
   R₁₆ is amino, hydroxy, C1-C12 alkoxy, C1-C12 substituted alkoxy, C1-C12 alkylamino, C1-C12 substituted alkylamino C1-C12 dialkylamino or C1-C12 substituted dialkylamino; or
further defined by the formula:

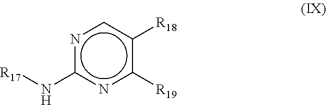

wherein:
   R₁₇, R₁₈, and R₁₉ are each independently C1-C12 heteroaryl or C1-C12 substituted heteroaryl; or
further defined by the formula:

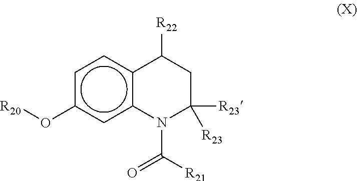

wherein:
   R₂₀, R₂₁, R₂₂, R₂₃, and R₂₃′ are each independently C1-C8 alkyl or C1-C8 substituted alkyl; or
further defined by the formula:

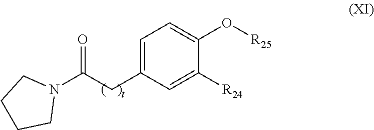

wherein:
   t is 1, 2, or 3; and
   R₂₄ and R₂₅ are each independently C1-C8 alkyl or C1-C8 substituted alkyl; or
further defined by the formula:

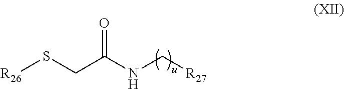

wherein:
   u is 1, 2, 3, 4, or 5; and
   R₂₆ and R₂₇ are each independently C6-C12 aryl, C6-C12 substituted aryl, C₁-C12 heteroaryl or C1-C12 substituted heteroaryl; or
further defined by the formula:

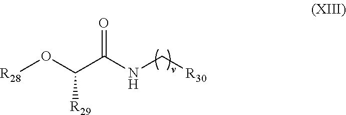

wherein:
   v is 1, 2, 3, 4, or 5; and
   R₂₈, R₂₉, and R₃₀ are each independently C1-C12 alkyl, C1-C12 substituted alkyl, C6-C12 aryl, C6-C12 substituted aryl, C1-C12 heteroaryl or C1-C12 substituted heteroaryl; and/or
(L) the compound is defined as shown in Table 1.

In some embodiments, the disease or disorder is associated with increased reactive oxygen species production. In other embodiments, the disease or disorder is associated with decreased reactive oxygen species production. In other embodiments, the disease or disorder is a disease or disorder may be treated with a change in reactive oxygen species.

In some embodiments, the change in reactive oxygen species is an increase in reactive oxygen species. In other embodiments, the change in reactive oxygen species is a decrease in reactive oxygen species.

In still other aspects, the present disclosure provides methods of increasing reactive oxygen species in a patient comprising administering to the patient a compound that binds to the NQ site in the respiratory complex III. In some embodiments, the compound that binds to the NQ site changes the conformation of phenylalanine 90 of the respiratory complex III. In some embodiments, the compound hinders the electron transfer between heme b_{L} and heme b_{H}. In some embodiments, the compound is further defined as:

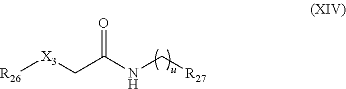

wherein:
X₃ is O, S, or NRₐ, wherein Rₐ is hydrogen, C1-C6 alkyl, or C1-C6 substituted alkyl;
u is 1, 2, 3, 4, or 5; and
R₂₆ and R₂₇ are each independently C6-C12 aryl, C6-C12 substituted aryl, C1-C12 heteroaryl or C1-C12 substituted heteroaryl;
   or a pharmaceutically acceptable salt thereof.

In some embodiments, the compound is further defined as:

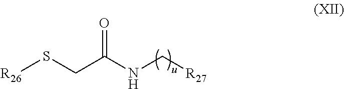

wherein:
u is 1, 2, 3, 4, or 5; and
R₂₆ and R₂₇ are each independently C6-C12 aryl, C6-C12 substituted aryl, C1-C12 heteroaryl or C1-C12 substituted heteroaryl;
   or a pharmaceutically acceptable salt thereof.

In some embodiments, the compound is further defined as:

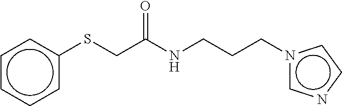

or a pharmaceutically acceptable salt thereof.

In another aspects, the present disclosure provides methods of decreasing reactive oxygen species in a patient comprising administering to the patient a compound that binds to the NQ site in the respiratory complex III. In some embodiments, the compound that binds to the NQ site changes the conformation of phenylalanine 90 of the respiratory complex III. In some embodiments, the compound enhances the electron transfer between heme b_{L} and heme b_{H}. In some embodiments, the compound is further defined as:

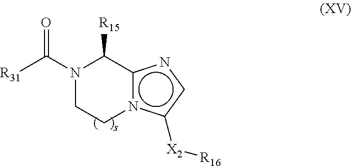

wherein:
R₃₁ is C1-C12 heteroaryl or C1-C12 substituted heteroaryl;
s is 0, 1, 2, or 3;
X₂ are C1-C6 alkanediyl or C1-C6 substituted alkanediyl;
R₁₅ is C1-C8 alkyl or C1-C8 substituted alkyl; and
R₁₆ is amino, hydroxy, C1-C12 alkoxy, C1-C12 substituted alkoxy, C1-C12 alkylamino, C1-C12 substituted alkylamino C1-C12 dialkylamino or C1-C12 substituted dialkylamino;
   or a pharmaceutically acceptable salt thereof.

In some embodiments, the compound is further defined as:

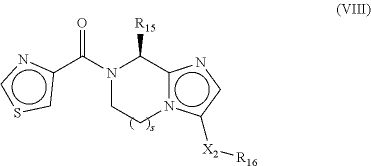

wherein:
s is 0, 1, 2, or 3;
X₂ are C1-C6 alkanediyl or C1-C6 substituted alkanediyl;
R₁₅ is C1-C8 alkyl or C1-C8 substituted alkyl; and
R₁₆ is amino, hydroxy, C1-C12 alkoxy, C1-C12 substituted alkoxy, C1-C12 alkylamino, C1-C12 substituted alkylamino C1-C12 dialkylamino or C1-C12 substituted dialkylamino;
   or a pharmaceutically acceptable salt thereof.

In some embodiments, the compound is further defined as:

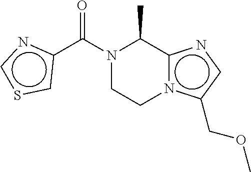

or a pharmaceutically acceptable salt thereof.

In some embodiments, the disease or disorder is chronic inflammation. In other embodiments, the disease or disorder is an autoimmune disease such as diabetes, rheumatoid arthritis, or lupus. In other embodiments, the disease or disorder is cancer. In other embodiments, the disease or disorder is an infectious disease. In some embodiments, the infectious disease is a bacterial infection. In other embodiments, the infectious disease is a viral infection. In other embodiments, the infectious disease is a fungal infection. In other embodiments, the disease or disorder is fibrotic disease. In some embodiments, the fibrotic disease is pulmonary fibrosis. In other embodiments, the fibrotic disease is diabetic nephropathy. In other embodiments, the fibrotic disease is liver fibrosis. In other embodiments, the disease or disorder is neurological disorder. In some embodiments, the neurological disorder is schizophrenia. In other embodiments, the neurological disorder is Alzheimer's disease. In other embodiments, the neurological disorder is Parkinson's disease. In other embodiments, the neurological disorder is amyotrophic lateral sclerosis (ALS). In other embodiments, the disease or disorder is a cardiovascular disease or disorder. In some embodiments, the cardiovascular disease or disorder is atherosclerosis. In other embodiments, the cardiovascular disease or disorder is hypertension. In other embodiments, the cardiovascular disease or disorder is restenosis. In other embodiments, the cardiovascular disease or disorder is ischemia/reperfusion injury. In other embodiments, the disease or disorder results in sensory impairment. In some embodiments, the sensory impairment is ocular disease. In other embodiments, the sensory impairment is hearing loss.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the disclosure will become apparent to those skilled in the art from this detailed description. Note that simply because a particular compound is ascribed to one particular generic formula doesn't mean that it cannot also belong to another generic formula.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein. The patent or application file contains at least one drawing executed in color. Copies of this patent or patent application publication with color drawing(s) will be provided by the Office upon request and payment of the necessary fee.

FIG. 1 shows The bc₁ complex showing all 4 redox centers in each monomer: Fe₂S₂, heme c₁, heme b_{L}, and heme b_{H}. Native ligands are shown: ubiquinol (UQH₂) and ubiquinone (UQ). Distances between corresponding redox centers or ligands are displayed in Angstrom.

FIG. 2 shows the bc₁ homodimer complex (PDB 1NTZ) embedded in the membrane. Different binding sites are visualized, including the Qₒ, Qᵢ, and NQ sites. Water molecules are displayed as red spheres. In the right inset, the NQ site entrance is displayed. In the left inset, the NQ site is shown as a solid surface with the lining Tyr95 residue displayed in red. Phe90 and Phe91 are also displayed as yellow and orange licorice residues, respectively.

FIGS. 3A-D show the virtual screening results of the ZINC database against the NQ, Qₒ, and Qᵢ binding sites. (A) Results of screening 1,489,806 compounds and the native ligands (UQ and UQH₂) against the NQ site. The dashed red line shows the binding energies of the native ligands. (B) Screening results against the NQ site in the fixed mode (upper plot) and flexible mode (middle plot). The difference in binding energies between fixed and flexible modes is shown in the lower plot. (C) Screening results against the Qₒ site. The dashed line indicates the binding energy of the native ligand. (D) Screening results against the Qᵢ site. The dashed line indicates the binding energy of the native ligand.

FIG. 4 shows the chemical structures of the purchased 14 ligands.

FIG. 5 shows the cell viability results for the 14 screened ligands.

FIG. 6 shows the cell viability results for the sequential treatment versus regular treatment with MH-200102 or positive controls.

FIGS. 7A-C show (Top) a plot of the total ROS fold intensity of the three lead hit compounds against the negative control (i.e., DMSO). Data are shown as mean fold change±SD. The results were evaluated using Student's t-test, and differences were considered significant at the **P<0.05 or ***P<0.005 or ****P<0.0001 level. (Bottom) Representative fluorescent images where the green fluorescence indicates the cellular total ROS levels and the nuclei are labeled in blue fluorescence.

FIGS. 8A-C show (Top) a plot of the mitochondrial superoxide anion fold intensity of the lead hit compounds against the negative control (i.e., DMSO). Data are shown as mean fold change±SD. The results were evaluated using Student's t-test, and differences were considered significant at the *P<0.05 or **P<0.01 or ***P<0.001 or ****P<0.0001 level. (Bottom) Representative fluorescent images where the red fluorescence indicates the mitochondrial superoxide anion levels and the nuclei are labeled in blue fluorescence.

FIG. 9 shows (Top) A plot of the mitochondrial superoxide anion fold intensity of the four lead hit compounds against the negative control (i.e., DMSO, or DMSO, and either MYX or ANT). Data are shown as mean fold change±SD. The results were evaluated using Student's t-test, and differences were considered significant at the *P<0.1 or **P<0.01 or ***P<0.001 or ****P<0.0001 level. (Bottom) Representative fluorescent images where the red fluorescence indicates the mitochondrial superoxide anion levels.

FIG. 10 shows (Upper plot) Electron transfer through-space distance between the heme b_{L}, Phe90, and heme b_{H} for the ligand-free monomer and for both MH-200102 or MH01015 docked monomers. Lower insets show the docked MH-200102 and MH-01015 conformation relative to Tyr95 (red) and the phenylamine dimer (Phe91-Phe90).

FIGS. 11A-C show (A) Normal ET state (the Q-cycle) where Phe90 exists in the ON conformation. (B) A malfunctioned ET state where Phe90 exists in the OFF conformation due to the bound ROS up-regulator (UP) at the NQ site, leading to increased ROS production. (C) Stabilized ET state where Phe90 exists in the ON conformation due to the bound ROS down-regulator drug (DOWN) at the NQ site. Red and blue arrows show the forward ET pathways for the two electrons of the UQH₂, while orange arrows show the reverse ET pathway.

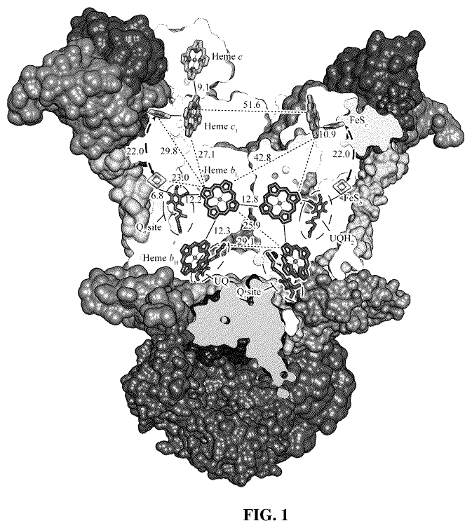

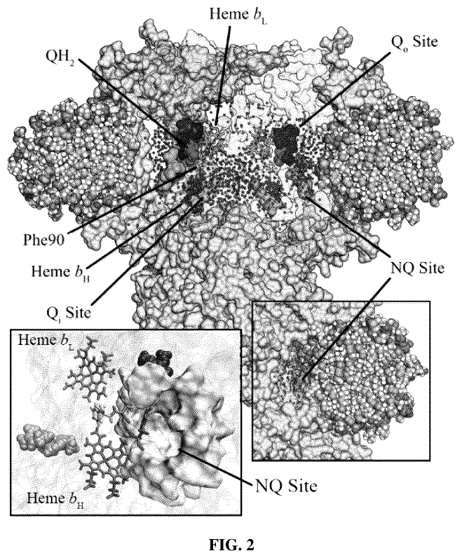

### DESCRIPTION OF ILLUSTRATIVE EMBODIMENTS

Described herein are methods for modulating the levels of reactive oxygen species (ROS). The present methods involve the use of certain compounds that bind to respiratory complex III at the Non-Q (NQ) binding site (NQ-binding site). The NQ-binding site extends up close to phenylanaine (Phe90) residue, an internal switch that regulates electron transfer (ET) between heme b_{L} and heme b_{H} of the low potential arm of the respiratory complex III. Compounds that bind to the NQ binding site of the respiratory complex III may be used to modulate the conformation of the Phe90 residue and thus control the ET reaction between the heme b_{L} and heme b_{H} redox pairs, leading to the regulation of the mitochondrial production levels of e or more reactive oxygen species. Alternatively, in other embodiments, the present methods lead to an activation of respiratory complex III, which corresponds with a reduction in ROS levels. In some embodiments, the present disclosure provides the use of compounds that may facilitate both elevation or reduction in ROS levels. That the present methods can be used to achieve elevated or reduced ROS levels makes the present disclosure useful for a wider variety of applications than other corresponding methods known in the art. For example, the present methods for ROS modulation may be useful or have benefits in anticancer, antibiotic, anti-inflammatory, or neuroprotective contexts in comparison to methods known in the art. In particular, the present compounds may be used as therapeutic agents for multiple diseases including, but not limited to, cancer, musculoskeletal disorders such as rheumatoid arthritis and osteoarthritis, cardiovascular diseases such as ischemia and hypertension, and neurodegenerative diseases such as Alzheimer's and Parkinson's diseases, inflammatory disorders, and infectious diseases. In some embodiments, the present disclosure provides methods that facilitate a greater level of control over ROS levels than methods known in the art. These and other aspects of the present disclosure are described in the claims and the following sections.

### I. COMPOUNDS OF THE PRESENT DISCLOSURE

Compounds that may be used according to the present disclosure are shown, for example, above, in the summary of the invention section, and in the claims below. They may be made using standard methods that can be further modified and optimized using the principles and techniques of organic chemistry as applied by a person skilled in the art. Such principles and techniques are taught, for example, in Smith, March's Advanced Organic Chemistry: Reactions, Mechanisms, and Structure, (2013), which is incorporated by reference herein. In addition, the synthetic methods may be further modified and optimized for preparative, pilot- or large-scale production, either batch or continuous, using the principles and techniques of process chemistry as applied by a person skilled in the art. Such principles and techniques are taught, for example, in Anderson, Practical Process Research & Development—A Guide for Organic Chemists (2012), which is incorporated by reference herein.

All of the present methods may in some embodiments be used for the prevention and treatment of one or more diseases or disorders discussed herein or otherwise. In some embodiments, one or more of the compounds characterized or exemplified herein as an intermediate, a metabolite, and/or prodrug, may nevertheless also be useful for the prevention and treatment of one or more diseases or disorders. As such unless explicitly stated to the contrary, all the compounds of the present invention are deemed “active compounds” and “therapeutic compounds” that are contemplated for use as active pharmaceutical ingredients (APIs). Actual suitability for human or veterinary use is typically determined using a combination of clinical trial protocols and regulatory procedures, such as those administered by the Food and Drug Administration (FDA). In the United States, the FDA is responsible for protecting the public health by assuring the safety, effectiveness, quality, and security of human and veterinary drugs, vaccines and other biological products, and medical devices.

In some embodiments, the compounds disclosed for use in the present methods have the advantage that they may be more efficacious than, be less toxic than, be longer acting than, be more potent than, produce fewer side effects than, be more easily absorbed than, more metabolically stable than, more lipophilic than, more hydrophilic than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

Compounds for use according to the presently disclosed methods may contain one or more asymmetrically-substituted carbon or nitrogen atom and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present invention can have the S or the R configuration.

Chemical formulas used to represent compounds for use according to the present methods will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

In addition, atoms making up the compounds for use according to the present methods are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In some embodiments, the compounds for use according to the present methods exist in salt or non-salt form. With regard to the salt form(s), in some embodiments the particular anion or cation forming a part of any salt form of a compound provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

Non-limiting examples of compounds disclosed for use herein include the compounds shown in Table 1.

TABLE 1

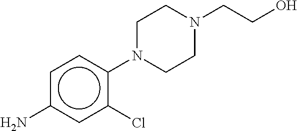

MH-U0206

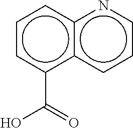

MH-01015

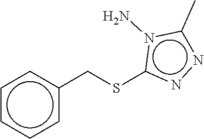

MH-U09509

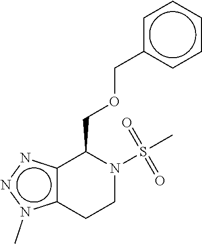

MH-U1014

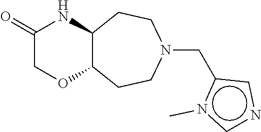

MH-U124012

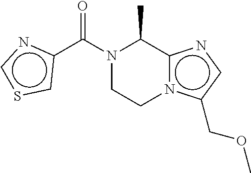

MH-131013

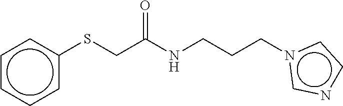

MH-200102

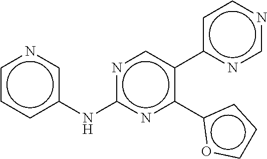

MH-D00101

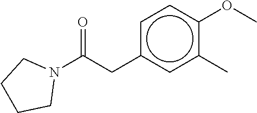

MH-D00008

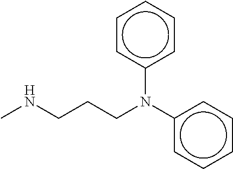

MH-N101010

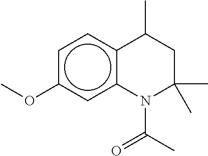

MH-N07507

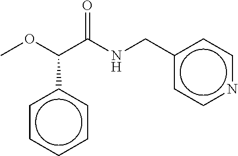

MH-N3103

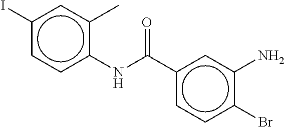

MH-04504

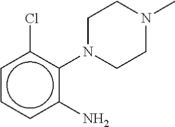

MH-D00005

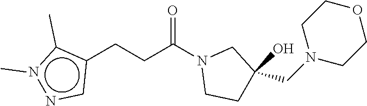

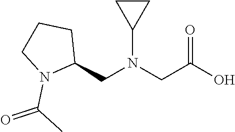 [SMILES: CC(=O)N1CCC[C@H]1CN(CC(=O)O)C1CC1]

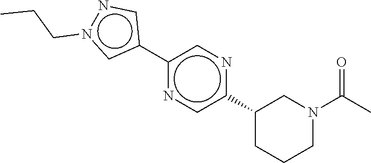

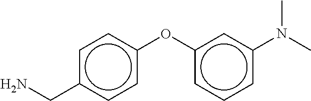

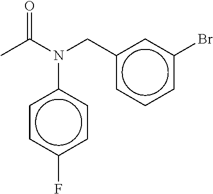

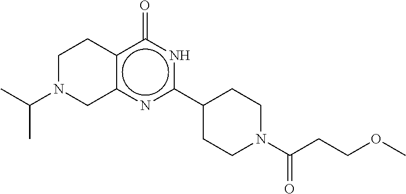

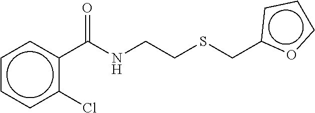

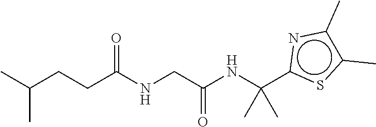

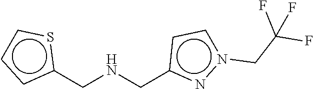

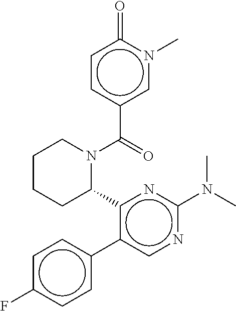

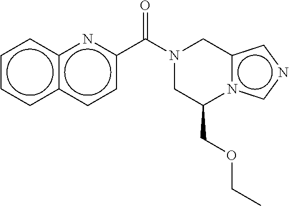

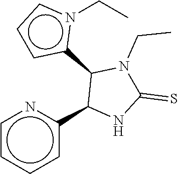

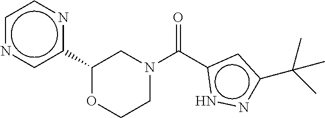

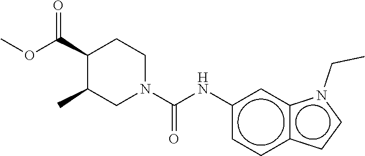

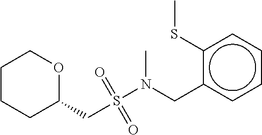

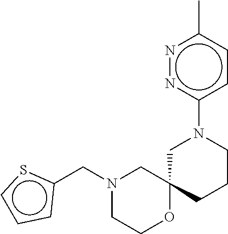

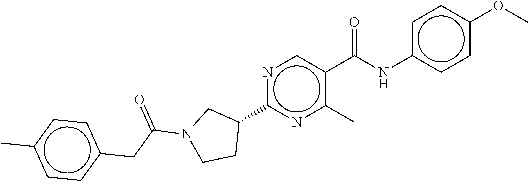

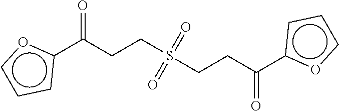

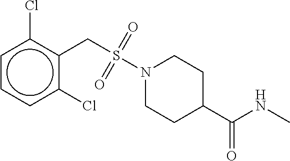

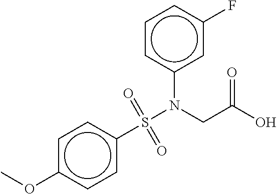

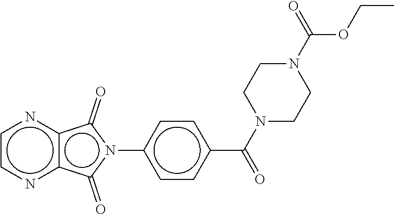

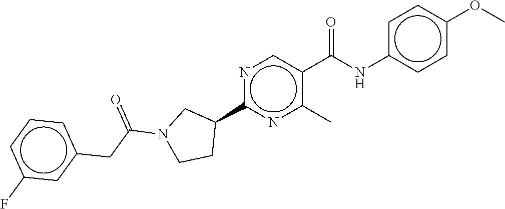

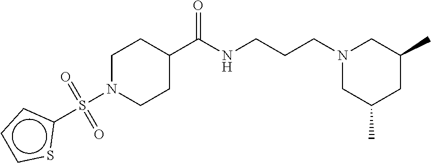

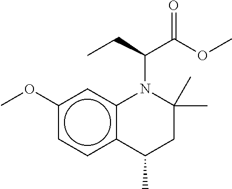

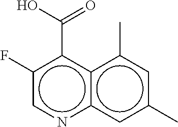

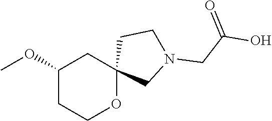 [SMILES: CO[C@H]1CCO[C@@]2(CCN(CC(=O)O)C2)C1]

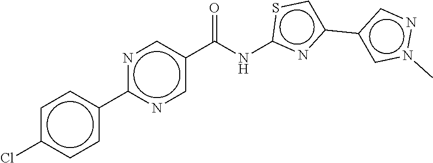

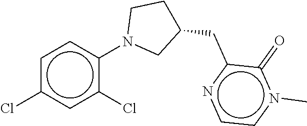

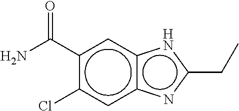

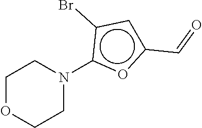

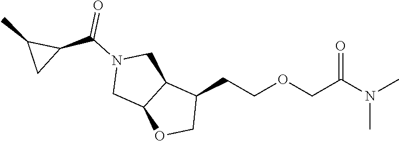

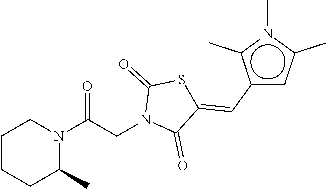

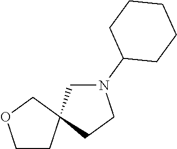 [SMILES: C1CCC(N2CC[C@]3(CCOC3)C2)CC1]

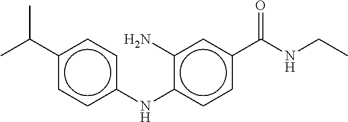

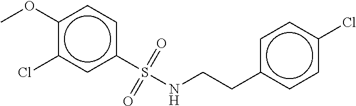

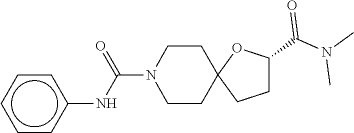

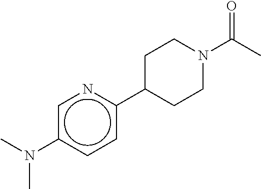

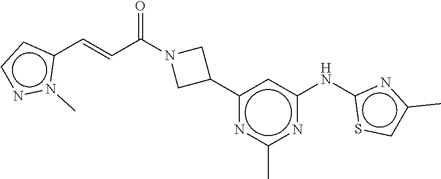

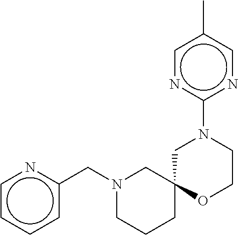

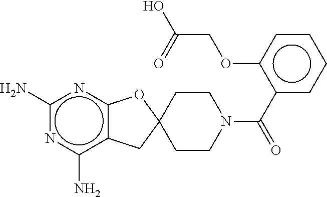

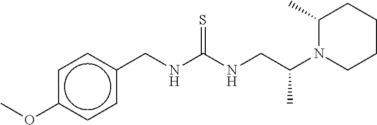

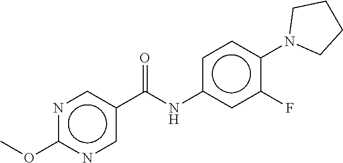

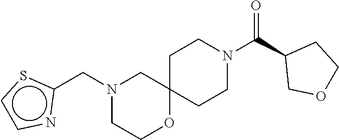

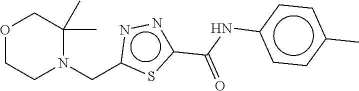

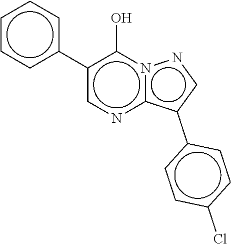

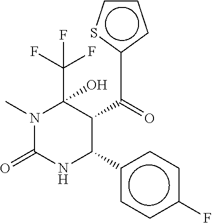

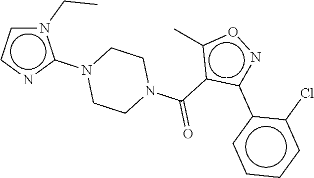

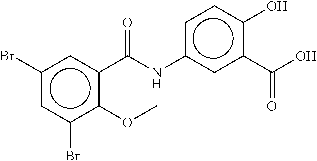

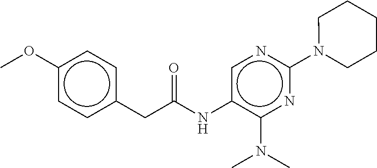

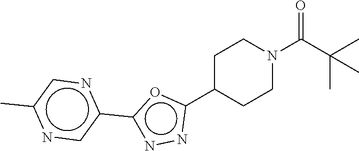

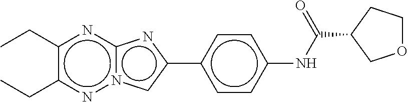

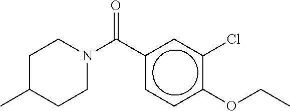

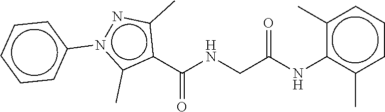

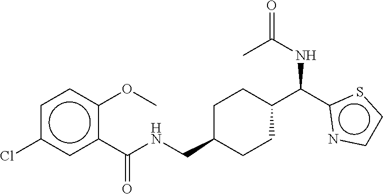

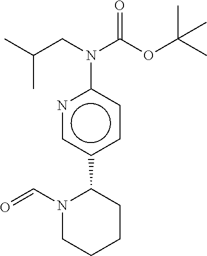

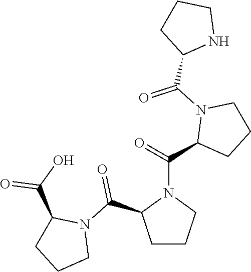 [SMILES: O=C(O)[C@@H]1CCCN1C(=O)[C@@H]1CCCN1C(=O)[C@@H]1CCCN1C(=O)[C@@H]1CCCN1]

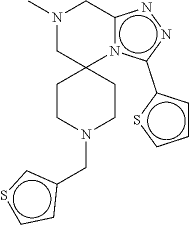

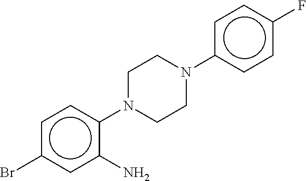

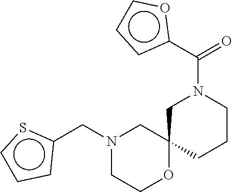

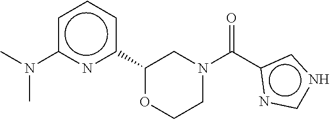

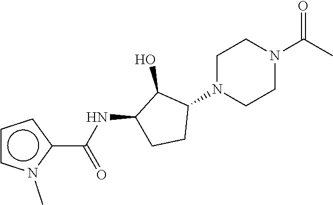

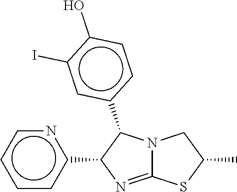

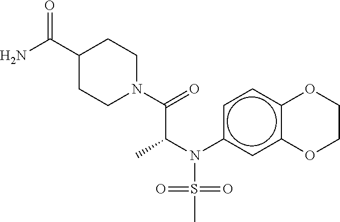

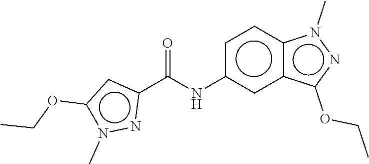

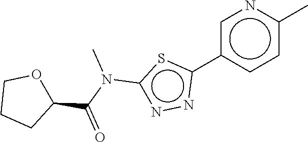

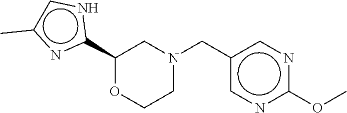

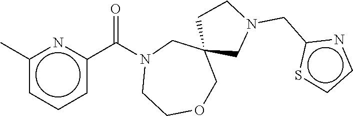

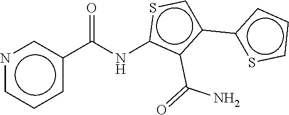

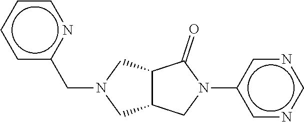

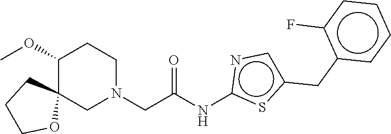

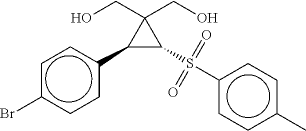

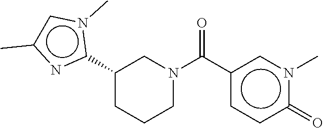

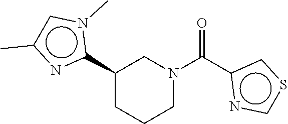

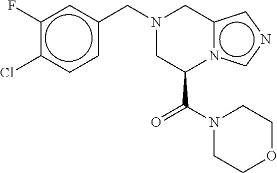

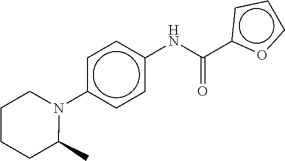

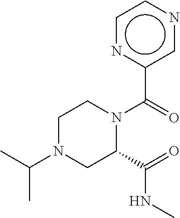

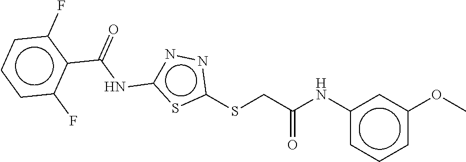

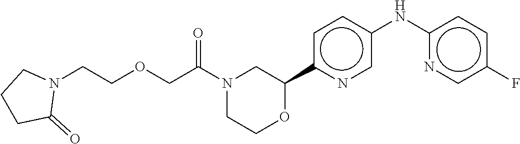

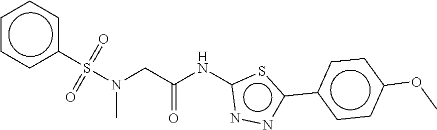

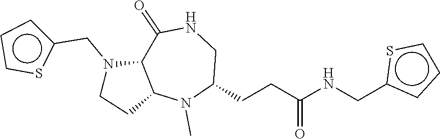

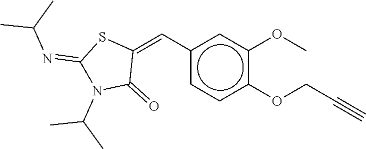

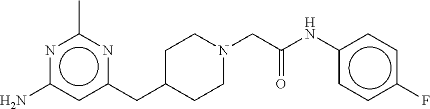

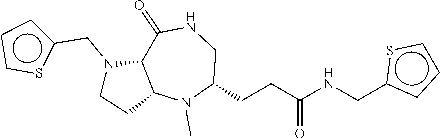

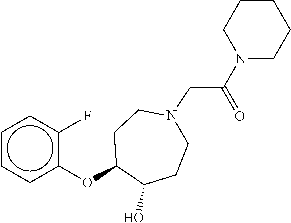

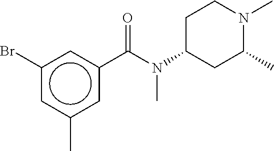

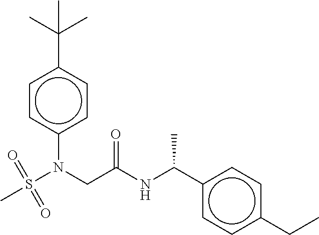

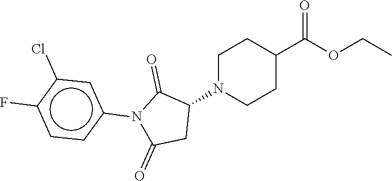

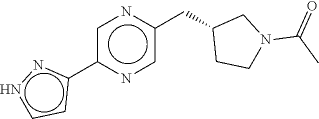

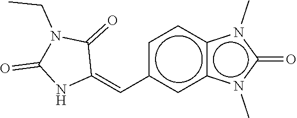

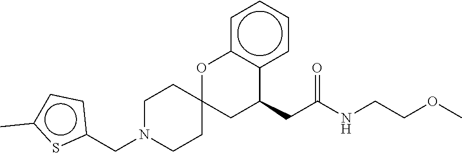

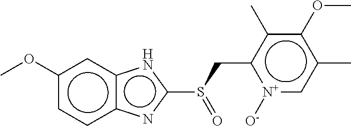

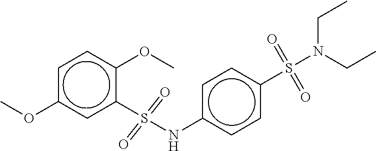

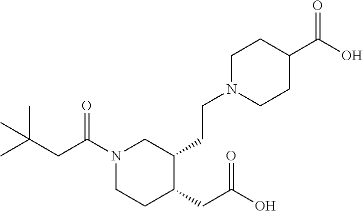 [SMILES: CC(C)(C)CC(=O)N1CC[C@@H](CC(=O)O)[C@@H](CCN2CCC(C(=O)O)CC2)C1]

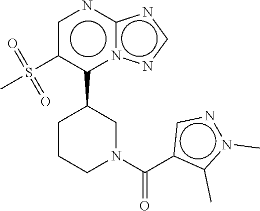

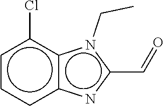

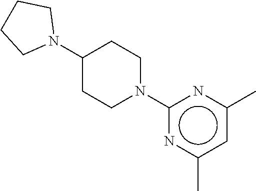

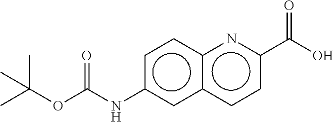

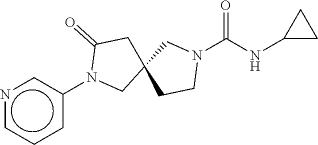

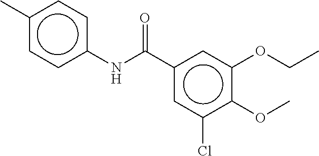

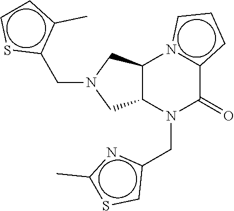

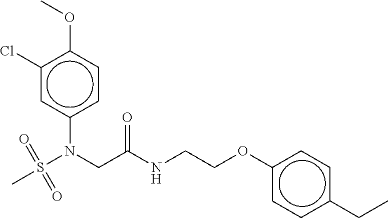

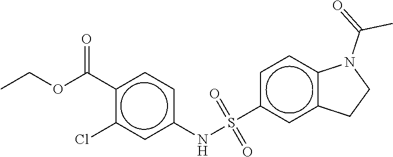

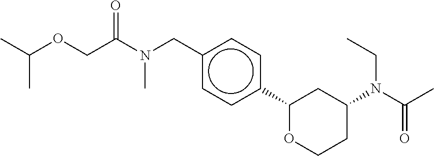

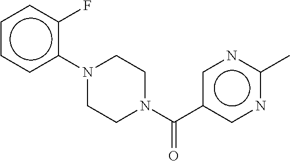

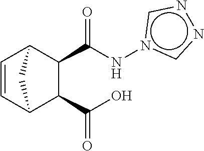

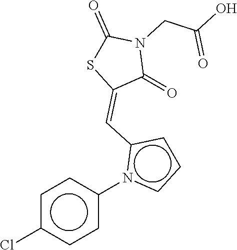

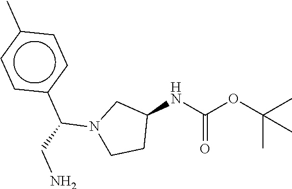

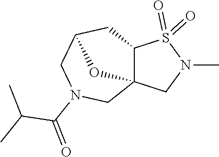 [SMILES: CC(C)C(=O)N1C[C@H]2C[C@H]3[C@](C1)(CN(C)S3(=O)=O)O2]

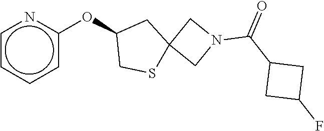

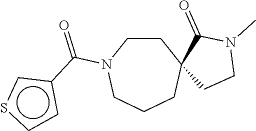

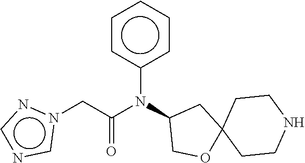

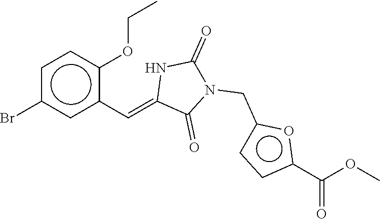

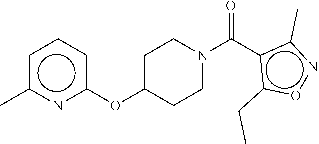

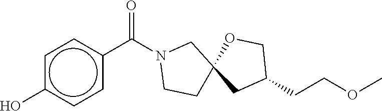

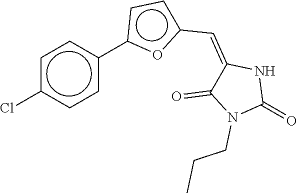

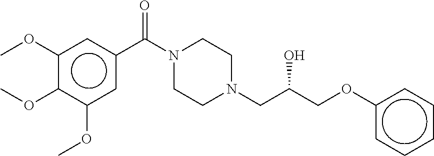

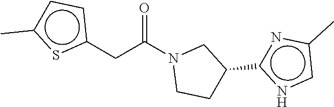

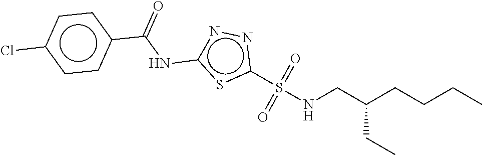

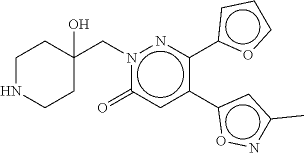

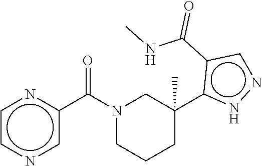

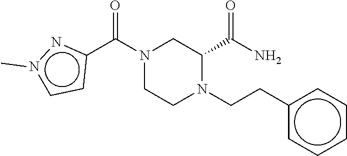

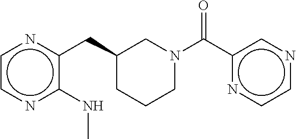

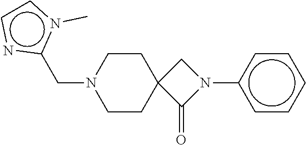

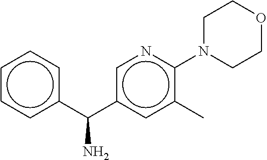

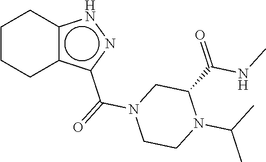

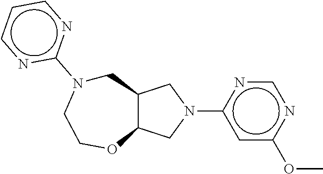

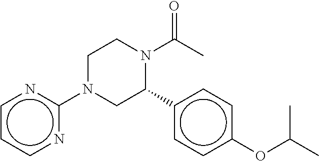

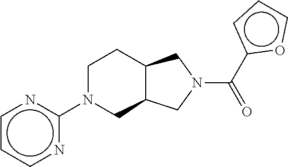

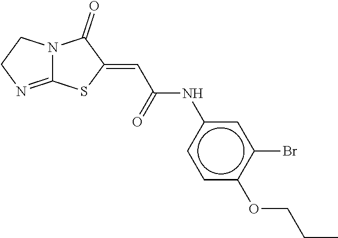

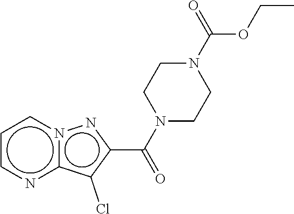

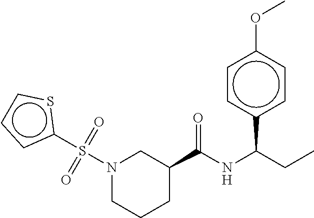

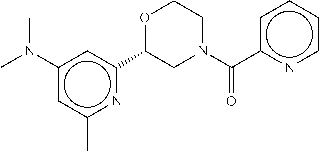

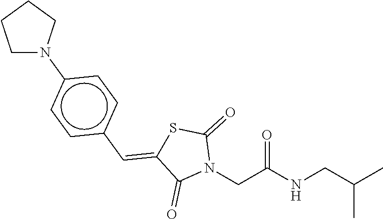

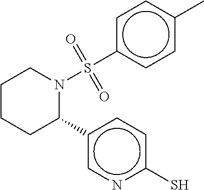

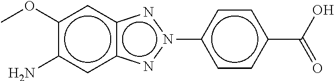

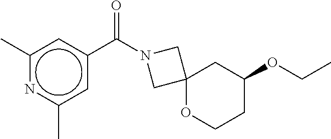

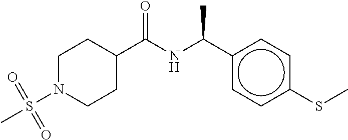

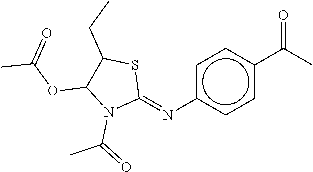

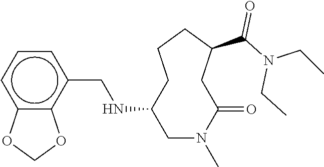

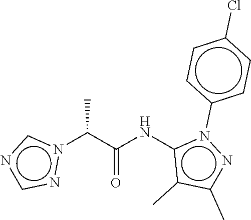

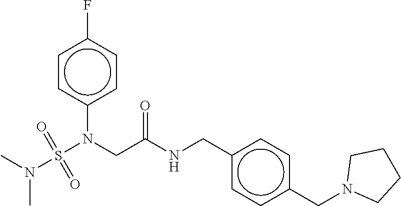

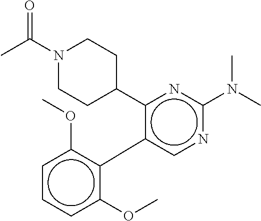

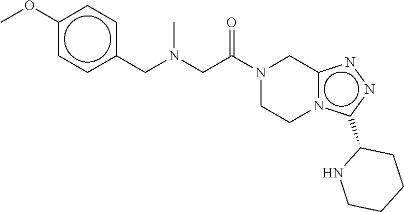

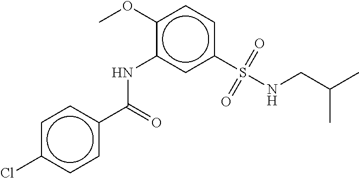

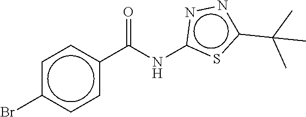

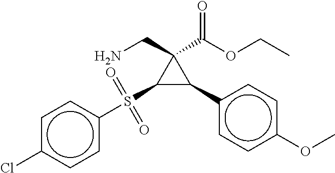

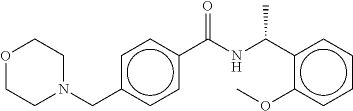

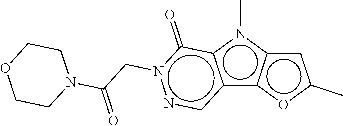

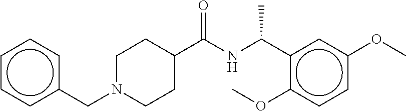

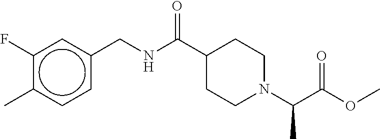

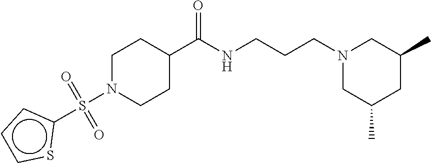

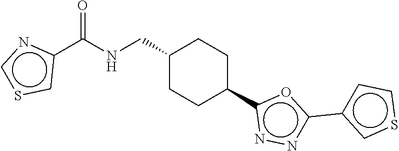

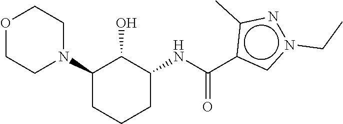

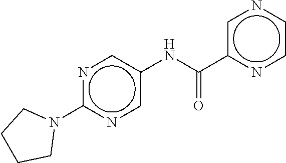

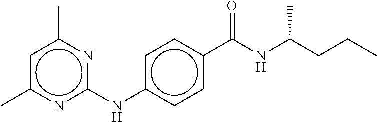

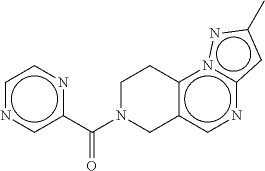

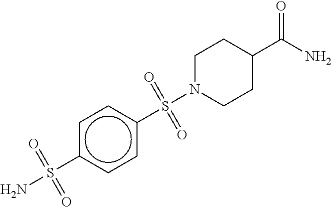

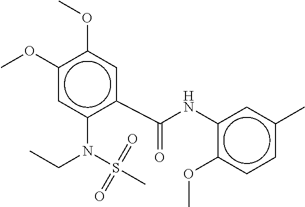

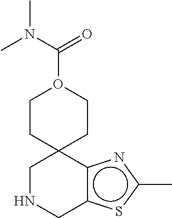

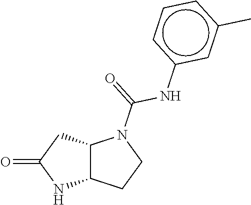

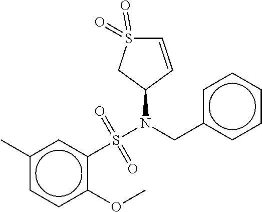

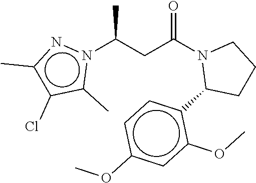

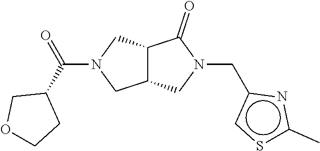

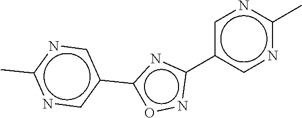

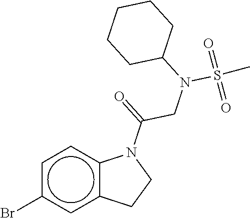

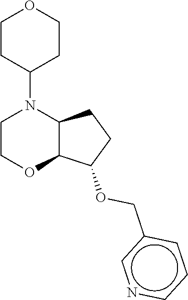

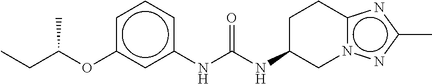

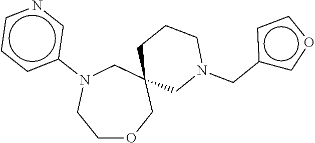

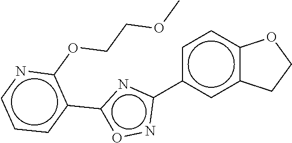

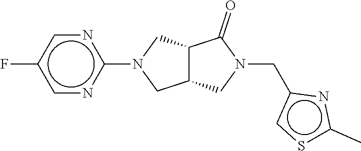

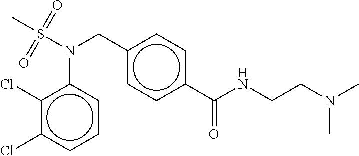

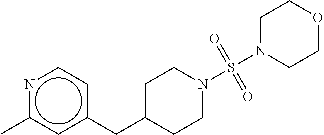

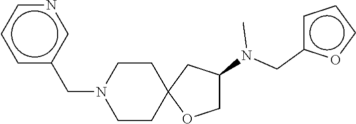

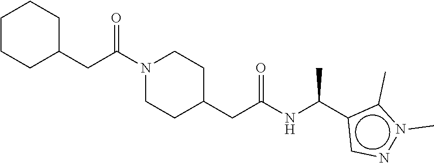

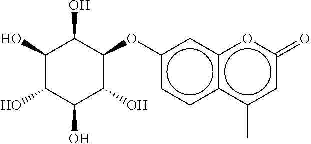

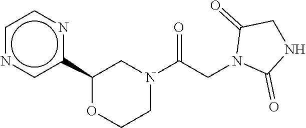

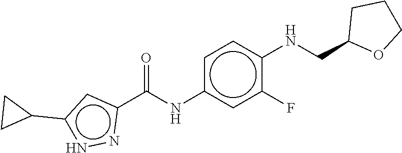

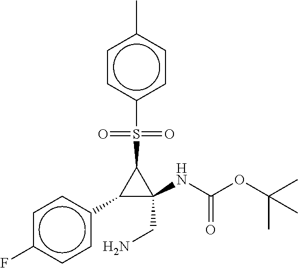

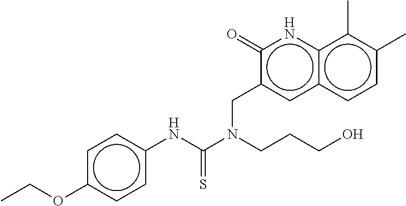

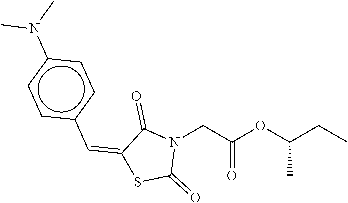

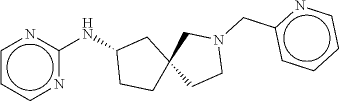

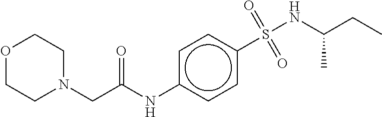

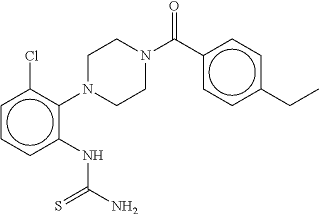

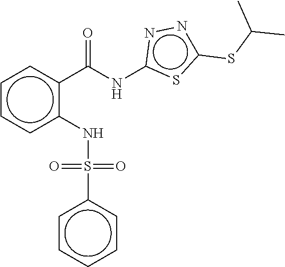

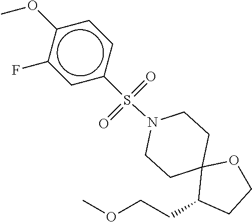

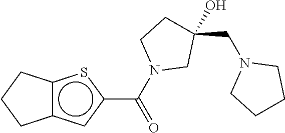

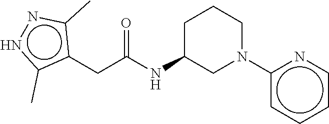

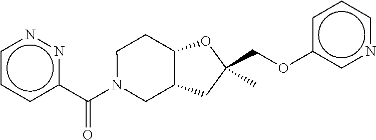

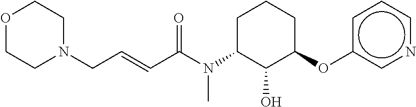

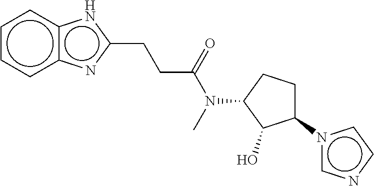

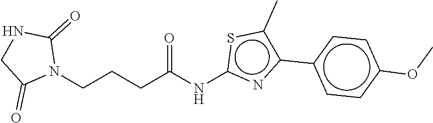

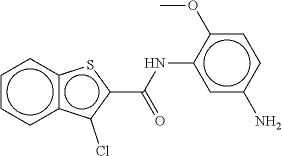

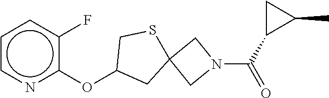

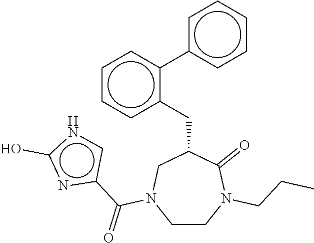

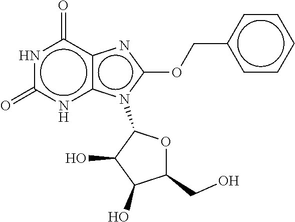

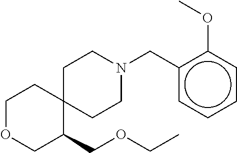

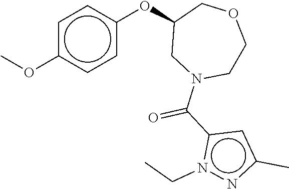

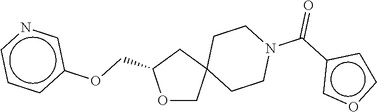

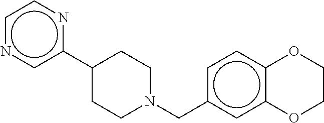

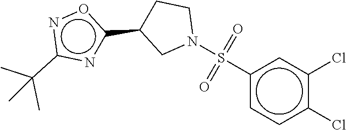

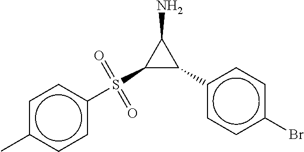

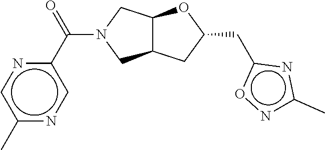

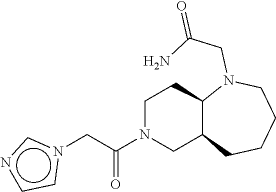

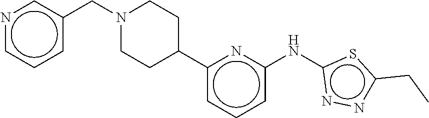

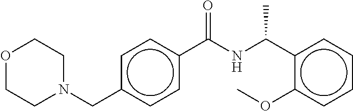

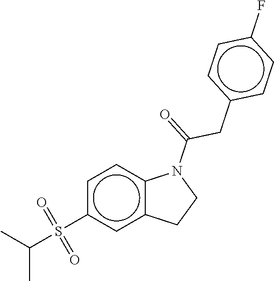

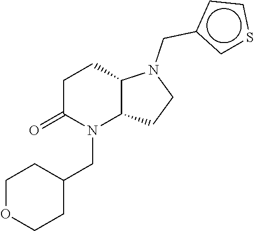

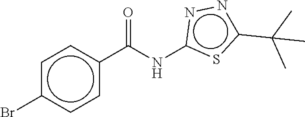

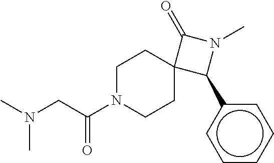

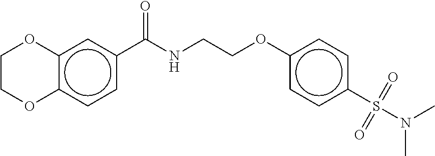

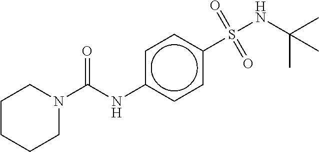

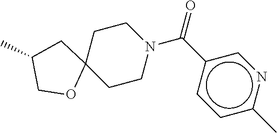

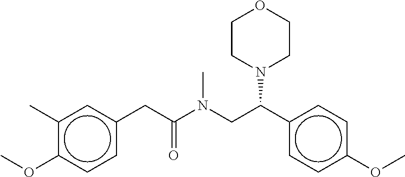

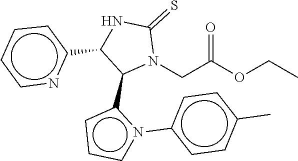

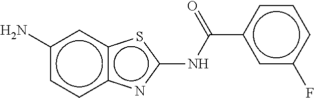

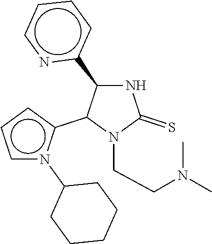

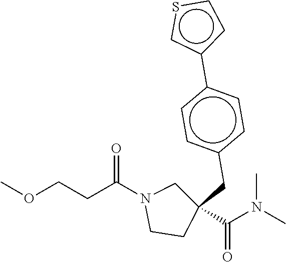

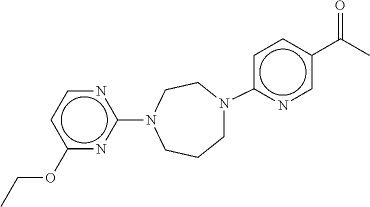

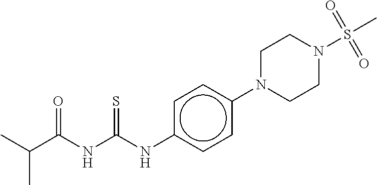

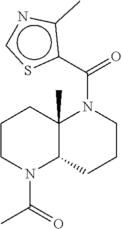

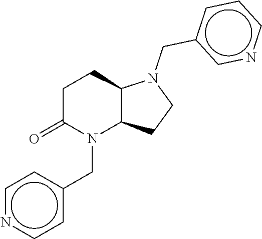

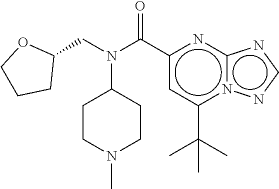

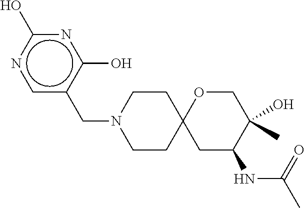

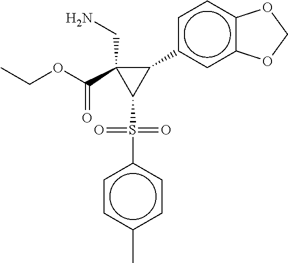

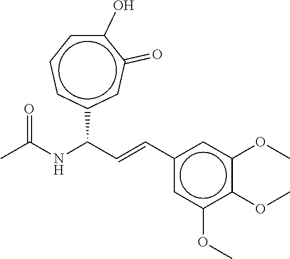

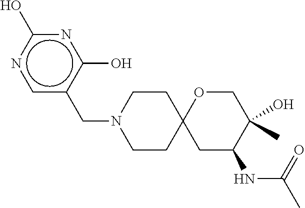

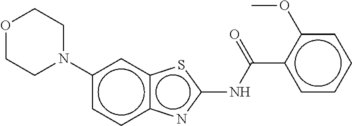

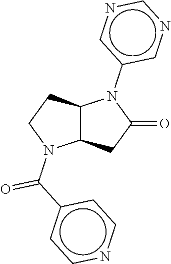

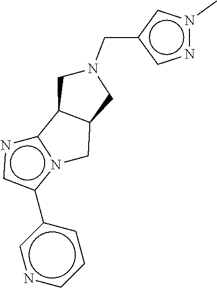

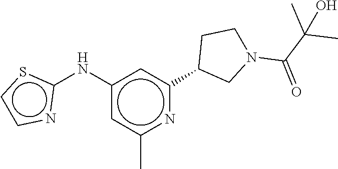

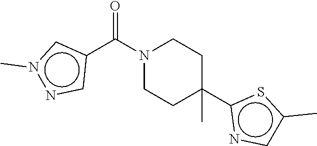

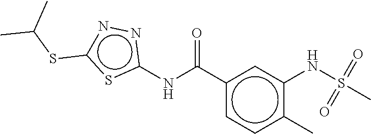

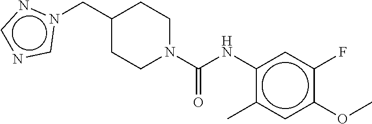

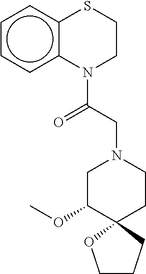

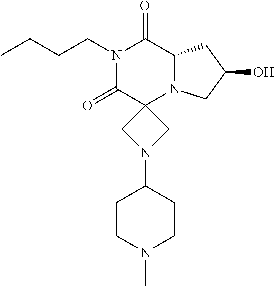 [SMILES: CCCCN1C(=O)[C@@H]2C[C@@H](O)CN2C2(CN(C3CCN(C)CC3)C2)C1=O]

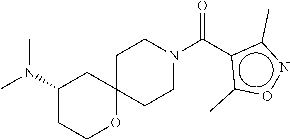

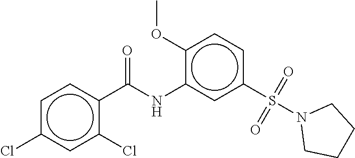

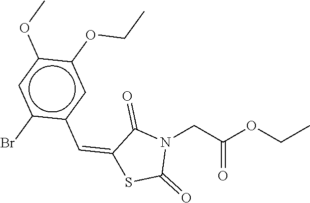

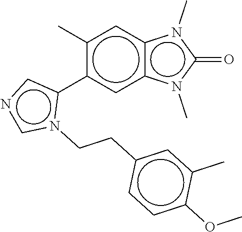

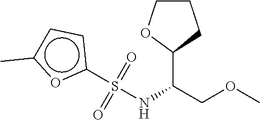

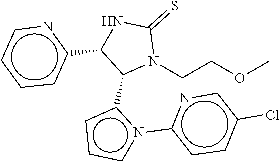

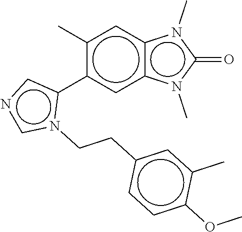

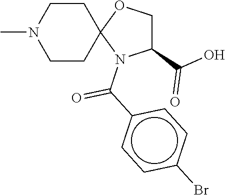

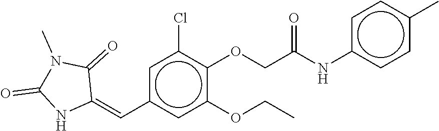

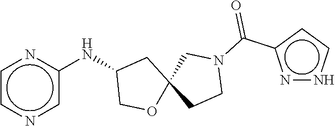

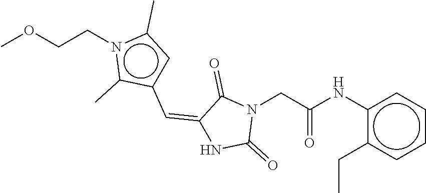

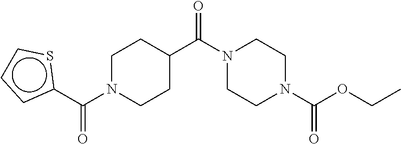

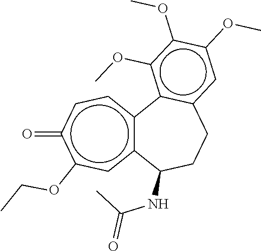

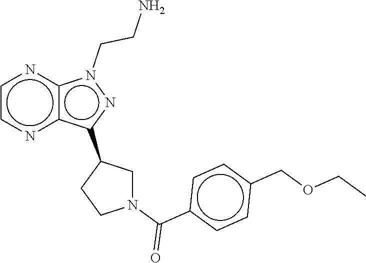

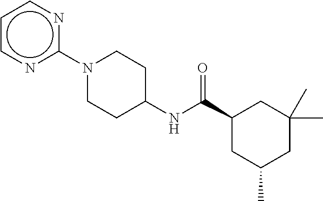

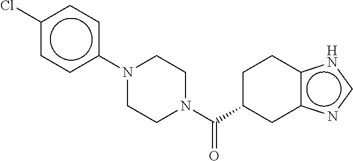

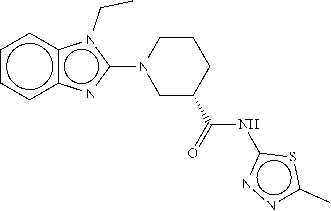

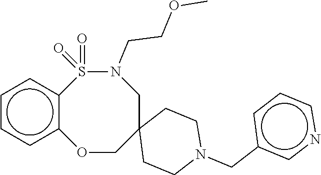

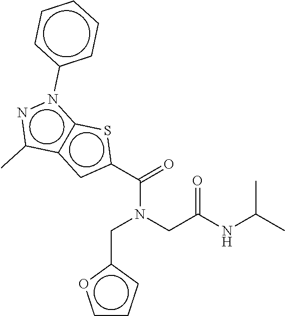

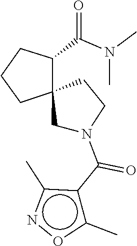

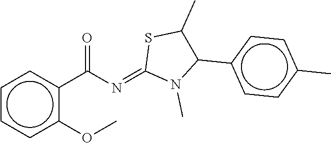

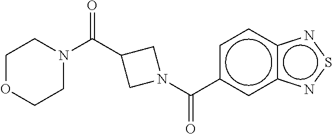

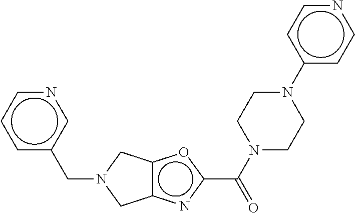

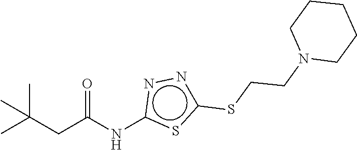

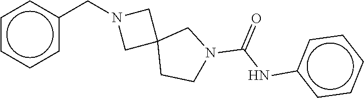

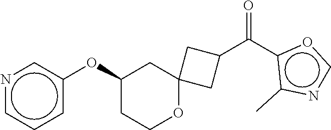

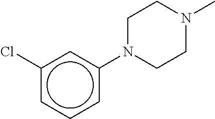

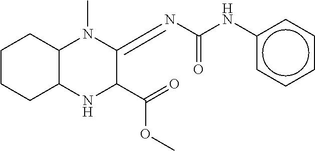

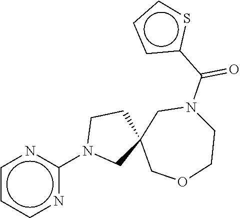

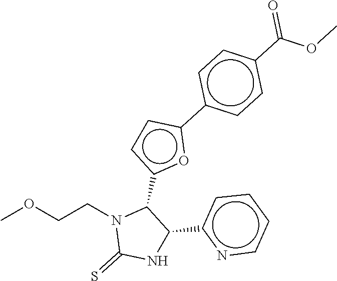

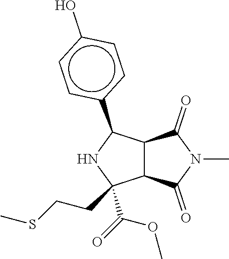

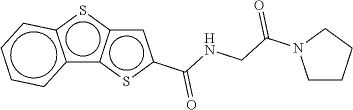

### II. Reactive Oxygen Species and Diseases Associated with Inflammation and/or Oxidative Stress

Inflammation is a biological process that provides resistance to infectious or parasitic organisms and the repair of damaged tissue. Inflammation is commonly characterized by localized vasodilation, redness, swelling, and pain, the recruitment of leukocytes to the site of infection or injury, production of inflammatory cytokines, such as TNF-α and IL-1, and production of reactive oxygen or nitrogen species, such as hydrogen peroxide, superoxide, and peroxynitrite. In later stages of inflammation, tissue remodeling, angiogenesis, and scar formation (fibrosis) may occur as part of the wound healing process. Under normal circumstances, the inflammatory response is regulated, temporary, and is resolved in an orchestrated fashion once the infection or injury has been dealt with adequately. However, acute inflammation can become excessive and life-threatening if regulatory mechanisms fail. Alternatively, inflammation can become chronic and cause cumulative tissue damage or systemic complications. Based at least on the evidence presented herein, the methods disclosed herein can be used in the treatment or prevention of inflammation or diseases, disorders, conditions, or infections (such as a viral, bacterial, or fungal infection) associated with inflammation, which are described in further detail below.

Many serious and intractable human diseases involve dysregulation of inflammatory processes, including diseases such as cancer, atherosclerosis, and diabetes, which were not traditionally viewed as inflammatory conditions. In the case of cancer, the inflammatory processes are associated with tumor formation, progression, metastasis, and resistance to therapy. Atherosclerosis, long viewed as a disorder of lipid metabolism, is now understood to be primarily an inflammatory condition, with activated macrophages playing an important role in the formation and eventual rupture of atherosclerotic plaques. Activation of inflammatory signaling pathways has also been shown to play a role in the development of insulin resistance, as well as in the peripheral tissue damage associated with diabetic hyperglycemia. Excessive production of reactive oxygen species and reactive nitrogen species, such as superoxide, hydrogen peroxide, nitric oxide, and peroxynitrite, is a hallmark of inflammatory conditions. Evidence of dysregulated peroxynitrite production has been reported in a wide variety of diseases (Szabo et al., 2007; Schulz et al., 2008; Forstermann, 2006; Pall, 2007).

Autoimmune diseases such as rheumatoid arthritis, lupus, psoriasis, and multiple sclerosis involve inappropriate and chronic activation of inflammatory processes in affected tissues, arising from dysfunction of self vs. non-self recognition and response mechanisms in the immune system. In neurodegenerative diseases such as Alzheimer's and Parkinson's diseases, neural damage is correlated with activation of microglia and elevated levels of pro-inflammatory proteins, such as inducible nitric oxide synthase (iNOS). Chronic organ failure, such as renal failure, heart failure, liver failure, and chronic obstructive pulmonary disease, is closely associated with the presence of chronic oxidative stress and inflammation, leading to the development of fibrosis and eventual loss of organ function. Oxidative stress in vascular endothelial cells, which line major and minor blood vessels, can lead to endothelial dysfunction and is believed to be an important contributing factor in the development of systemic cardiovascular disease, complications of diabetes, chronic kidney disease and other forms of organ failure, and a number of other aging-related diseases, including degenerative diseases of the central nervous system and the retina.

Many other disorders involve oxidative stress and inflammation in affected tissues, including inflammatory bowel disease; inflammatory skin diseases; mucositis and dermatitis related to radiation therapy and chemotherapy; ocular or eye diseases, such as uveitis, glaucoma, macular degeneration, and various forms of retinopathy; transplant failure and rejection; ischemia-reperfusion injury; chronic pain; degenerative conditions of the bones and joints, including osteoarthritis and osteoporosis; asthma and cystic fibrosis; liver fibrosis; seizure disorders; and neuropsychiatric conditions, including schizophrenia, depression, bipolar disorder, post-traumatic stress disorder, attention deficit disorders, autism-spectrum disorders, and eating disorders, such as anorexia nervosa. Dysregulation of inflammatory signaling pathways is believed to be a major factor in the pathology of muscle wasting diseases, including muscular dystrophy and various forms of cachexia.

A variety of life-threatening acute disorders also involve dysregulated inflammatory signaling, including acute organ failure involving the pancreas, kidneys, liver, or lungs, myocardial infarction or acute coronary syndrome, stroke, septic shock, trauma, severe burns, and anaphylaxis.

Many complications of infectious diseases also involve dysregulation of inflammatory responses. Although an inflammatory response can kill invading pathogens, an excessive inflammatory response can also be quite destructive and in some cases can be a primary source of damage in infected tissues. The balance between production and elimination of reactive oxygen species in a healthy cell or patient is known to be disrupted or disturbed when the host becomes infected with a microbe, such as a virus or bacteria (Li et al., 2017). There is also evidence that reactive oxygen species are relevant in the interaction of host and fungal pathogens, and in the intracellular processes of fungal pathogens (Hogan and Wheeler, 2014; Marschall & Tudzynski, 2016). Furthermore, an excessive inflammatory response can also lead to systemic complications due to overproduction of inflammatory cytokines, such as TNF-α and IL-1. This is believed to be a factor in mortality arising from severe influenza, severe acute respiratory syndrome, and sepsis.

In one aspect, the present disclosure provides methods of modulating the levels of reactive oxygen species. In some embodiments, the disclosure provides methods of elevating the levels of reactive oxygen species. In some embodiments, the present disclosure provides methods of reducing the level of reactive oxygen species. These properties are relevant to the treatment of a wide array of diseases and disorders involving oxidative stress and dysregulation of inflammatory processes, including cancer, complications from localized or total-body exposure to ionizing radiation, mucositis and dermatitis resulting from radiation therapy or chemotherapy, autoimmune diseases, cardiovascular diseases, including atherosclerosis or hypertension or ischemia-reperfusion injury or restenosis, acute and chronic organ failure, including renal failure and heart failure, respiratory diseases, diabetes and complications of diabetes, severe allergies, transplant rejection, graft-versus-host disease, neurodegenerative diseases, diseases of the eye and retina, acute and chronic pain, degenerative bone diseases, including osteoarthritis and osteoporosis, inflammatory bowel diseases, dermatitis and other skin diseases, sepsis, burns, seizure disorders, and neuropsychiatric disorders.

In another aspect, the present methods may be used for treating a subject having a condition such as ocular diseases. For example, uveitis, macular degeneration (both the dry form and wet form), glaucoma, diabetic macular edema, blepharitis, diabetic retinopathy, diseases and disorders of the corneal endothelium such as Fuchs endothelial corneal dystrophy, post-surgical inflammation, dry eye, allergic conjunctivitis and other forms of conjunctivitis are non-limiting examples of eye diseases that could be treated with the present methods.

In another aspect, the present methods may be used for treating a subject having a condition such as skin diseases or disorders. For example, dermatitis, including allergic dermatitis, atopic dermatitis, dermatitis due to chemical exposure, and radiation-induced dermatitis; thermal or chemical burns; chronic wounds including diabetic ulcers, pressure sores, and venous ulcers; acne; alopecia including baldness and drug-induced alopecia; other disorders of the hair follicle; epidermolysis bullosa; sunburn and its complications; disorders of skin pigmentation including vitiligo; aging-related skin conditions; post-surgical wound healing; prevention or reduction of scarring from skin injury, surgery, or burns; psoriasis; dermatological manifestations of autoimmune diseases or graft-versus host disease; prevention or treatment of skin cancer; disorders involving hyperproliferation of skin cells such as hyperkeratosis is a non-limiting example of skin diseases that could be treated with the present methods.

In another aspect, the present methods may be used for treating a subject having a condition caused by elevated levels of oxidative stress in one or more tissues. Oxidative stress results from abnormally high or prolonged levels of reactive oxygen species, such as superoxide, hydrogen peroxide, nitric oxide, and peroxynitrite (formed by the reaction of nitric oxide and superoxide). The oxidative stress may be accompanied by either acute or chronic inflammation. The oxidative stress may be caused by mitochondrial dysfunction, by activation of immune cells, such as macrophages and neutrophils, by acute exposure to an external agent, such as ionizing radiation or a cytotoxic chemotherapy agent (e.g., doxorubicin), by trauma or other acute tissue injury, by ischemia/reperfusion, by poor circulation or anemia, by localized or systemic hypoxia or hyperoxia, by elevated levels of inflammatory cytokines and other inflammation-related proteins, and/or by other abnormal physiological states, such as hyperglycemia or hypoglycemia.

In another aspect, the present methods may be used in preventing or treating tissue damage or organ failure, acute and chronic, resulting from oxidative stress exacerbated by inflammation. Examples of diseases that fall in this category include heart failure, liver failure, transplant failure and rejection, renal failure, pancreatitis, fibrotic lung diseases (cystic fibrosis, COPD, and idiopathic pulmonary fibrosis, among others), diabetes (including complications), atherosclerosis, ischemia-reperfusion injury, glaucoma, stroke, autoimmune disease, autism, macular degeneration, and muscular dystrophy. For example, in the case of autism, studies suggest that increased oxidative stress in the central nervous system may contribute to the development of the disease (Chauhan and Chauhan, 2006).

Evidence also links oxidative stress and inflammation to the development and pathology of many other disorders of the central nervous system, including psychiatric disorders, such as psychosis, major depression, and bipolar disorder; seizure disorders, such as epilepsy; pain and sensory syndromes, such as migraine, neuropathic pain, ocular disease, hearing loss, or tinnitus; and behavioral syndromes, such as the attention deficit disorders. See, e.g., Dickerson et al., 2007; Hanson et al., 2005; Kendall-Tackett, 2007; Lencz et al., 2007; Dudhgaonkar et al., 2006; Lee et al., 2007; Morris et al., 2002; Ruster et al., 2005; McIver et al., 2005; Sarchielli et al., 2006; Kawakami et al., 2006; Ross et al., 2003, which are all incorporated by reference herein. For example, elevated levels of inflammatory cytokines, including TNF, interferon-γ, and IL-6, are associated with major mental illness (Dickerson et al., 2007). Microglial activation has also been linked to major mental illness. Therefore, downregulating inflammatory cytokines and inhibiting excessive activation of microglia could be beneficial in patients with schizophrenia, major depression, bipolar disorder, autism-spectrum disorders, and other neuropsychiatric disorders.

Accordingly, in pathologies involving oxidative stress alone or oxidative stress exacerbated by inflammation, treatment may comprise administering to a subject a therapeutically effective amount of a compound according to the present methods, such as those described above or throughout this specification. Treatment may be administered preventively, in advance of a predictable state of oxidative stress (e.g., organ transplantation or the administration of radiation therapy to a cancer patient), or it may be administered therapeutically in settings involving established oxidative stress and inflammation. In some instances, such as a cancer patient receiving radiation therapy or chemotherapy (or both), present methods may be used both before and after the radiation or chemotherapy, or may be used in combination with the other therapies. Depending on the nature of the radiation therapy or chemotherapy, various combinations of pre-treatment, post-treatment, or concurrent use of the present methods may be utilized. The presently disclosed methods may prevent or reduce the severity of side effects associated with the radiation therapy or chemotherapy. Because such side effects may be dose-limiting, their reduction or prevention may allow higher or more frequent dosing of the radiation therapy or chemotherapy, resulting in greater efficacy. Alternatively, as shown herein, use of the presently disclosed methods in combination with the radiation therapy or chemotherapy may enhance the efficacy of a given dose of radiation or chemotherapy. In part, this combinatorial efficacy may result from inhibition of the activity of the pro-inflammatory transcription factor NF-κB by the compound of the invention. NF-κB is often chronically activated in cancer cells, and such activation is associated with resistance to therapy and promotion of tumor progression (e.g., Karin M, Nature. 2006 May 25; 441(7092):431-6; Aghajan et al., J Gastroenterol Hepatol. 2012 March; 27 Suppl 2:10-4). Other transcription factors that promote inflammation and cancer, such as STAT3 (e.g., He G and Karin M, Cell Res. 2011 January; 21(1):159-68; Grivennikov SI and Karin M, Cytokine Growth Factor Rev. 2010 February; 21(1):11-9), may also be inhibited by the compound of the invention.

The presently disclosed methods may be used to treat or prevent inflammatory conditions, such as sepsis, dermatitis, autoimmune disease, and osteoarthritis. The presently disclosed methods may also be used to treat or prevent inflammatory pain and/or neuropathic pain.

The presently disclosed methods may also be used to treat or prevent diseases, such as cancer, inflammation, Alzheimer's disease, Parkinson's disease, multiple sclerosis, autism, amyotrophic lateral sclerosis, Huntington's disease, autoimmune diseases, such as rheumatoid arthritis, lupus, Crohn's disease, and psoriasis, inflammatory bowel disease, all other diseases whose pathogenesis is believed to involve excessive production of either nitric oxide or prostaglandins, and pathologies involving oxidative stress alone or oxidative stress exacerbated by inflammation.

### III. PHARMACEUTICAL FORMULATIONS AND ROUTES OF ADMINISTRATION

In another aspect, for administration to a patient in need of such treatment, pharmaceutical formulations (also referred to as a pharmaceutical preparations, pharmaceutical compositions, pharmaceutical products, medicinal products, medicines, medications, or medicaments) comprise a therapeutically effective amount of the compounds disclosed herein formulated with one or more excipients and/or drug carriers appropriate to the indicated route of administration. In some embodiments, the compounds disclosed herein are formulated in a manner amenable for the treatment of human and/or veterinary patients. In some embodiments, formulation comprises admixing or combining one or more of the compounds disclosed herein with one or more of the following excipients: lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol. In some embodiments, e.g., for oral administration, the pharmaceutical formulation may be tableted or encapsulated. In some embodiments, the compounds may be dissolved or slurried in water, polyethylene glycol, propylene glycol, ethanol, corn oil, cottonseed oil, peanut oil, sesame oil, benzyl alcohol, sodium chloride, and/or various buffers. In some embodiments, the pharmaceutical formulations may be subjected to pharmaceutical operations, such as sterilization, and/or may contain drug carriers and/or excipients such as preservatives, stabilizers, wetting agents, emulsifiers, encapsulating agents such as lipids, dendrimers, polymers, proteins such as albumin, nucleic acids, and buffers.

Pharmaceutical formulations may be administered by a variety of methods, e.g., orally or by injection (e.g. subcutaneous, intravenous, and intraperitoneal). Depending on the route of administration, the compounds for use according to the present methods may be coated in a material to protect the compound from the action of acids and other natural conditions which may inactivate the compound. To administer the active compound by other than parenteral administration, it may be necessary to coat the compound with, or co-administer the compound with, a material to prevent its inactivation. In some embodiments, the active compound may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes.

The compounds for use according to the present methods may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

The compounds for use according to the present methods can be administered orally, for example, with an inert diluent or an assimilable edible carrier. The compounds and other ingredients may also be enclosed in a hard or soft-shell gelatin capsule, compressed into tablets, or incorporated directly into the patient's diet. For oral therapeutic administration, the compounds disclosed herein may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such pharmaceutical formulations is such that a suitable dosage will be obtained.

The therapeutic compound may also be administered topically to the skin, eye, ear, or mucosal membranes. Administration of the therapeutic compound topically may include formulations of the compounds as a topical solution, lotion, cream, ointment, gel, foam, transdermal patch, or tincture. When the therapeutic compound is formulated for topical administration, the compound may be combined with one or more agents that increase the permeability of the compound through the tissue to which it is administered. In other embodiments, it is contemplated that the topical administration is administered to the eye. Such administration may be applied to the surface of the cornea, conjunctiva, or sclera. Without wishing to be bound by any theory, it is believed that administration to the surface of the eye allows the therapeutic compound to reach the posterior portion of the eye. Ophthalmic topical administration can be formulated as a solution, suspension, ointment, gel, or emulsion. Finally, topical administration may also include administration to the mucosa membranes such as the inside of the mouth. Such administration can be directly to a particular location within the mucosal membrane such as a tooth, a sore, or an ulcer. Alternatively, if local delivery to the lungs is desired the therapeutic compound may be administered by inhalation in a dry-powder or aerosol formulation.

In some embodiments, it may be advantageous to formulate parenteral compositions in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical carrier. In some embodiments, the specification for the dosage unit forms of the invention are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient. In some embodiments, active compounds are administered at a therapeutically effective dosage sufficient to treat a condition associated with a condition in a patient. For example, the efficacy of a compound can be evaluated in an animal model system that may be predictive of efficacy in treating the disease in a human or another animal.

In some embodiments, the effective dose range for the therapeutic compound can be extrapolated from effective doses determined in animal studies for a variety of different animals. In some embodiments, the human equivalent dose (HED) in mg/kg can be calculated in accordance with the following formula (see, e.g., Reagan-Shaw et al., 2008, which is incorporated herein by reference):

$$HED ( mg / kg ) = Animal dose ( mg / kg ) \times ( Animal {K}_{m} / Human {K}_{m} )$$

Use of the Kₘ factors in conversion results in HED values based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are specific to each individual. Nonetheless, a calculated HED dose provides a general guide. Other factors affecting the dose include the physical and clinical state of the patient, the route of administration, the intended goal of treatment and the potency, stability and toxicity of the particular therapeutic formulation.

The actual dosage amount of a compound for use according to the present methods or composition comprising a compound for use according to the present methods administered to a patient may be determined by physical and physiological factors such as type of animal treated, age, sex, body weight, severity of condition, the type of disease being treated, previous or concurrent therapeutic interventions, idiopathy of the patient and on the route of administration. These factors may be determined by a skilled artisan. The practitioner responsible for administration will typically determine the concentration of active ingredient(s) in a composition and appropriate dose(s) for the individual patient. The dosage may be adjusted by the individual physician in the event of any complication.

In some embodiments, the therapeutically effective amount typically will vary from about 0.001 mg/kg to about 1000 mg/kg, from about 0.01 mg/kg to about 750 mg/kg, from about 100 mg/kg to about 500 mg/kg, from about 1 mg/kg to about 250 mg/kg, from about 10 mg/kg to about 150 mg/kg in one or more dose administrations daily, for one or several days (depending of course of the mode of administration and the factors discussed above). Other suitable dose ranges include 1 mg to 10,000 mg per day, 100 mg to 10,000 mg per day, 500 mg to 10,000 mg per day, and 500 mg to 1,000 mg per day. In some embodiments, the amount is less than 10,000 mg per day with a range of 750 mg to 9,000 mg per day.

In some embodiments, the amount of the active compound in the pharmaceutical formulation is from about 2 to about 98 weight percent. In some of these embodiments, the amount if from about 25 to about 60 weight percent.

Single or multiple doses of the agents are contemplated. Desired time intervals for delivery of multiple doses can be determined by one of ordinary skill in the art employing no more than routine experimentation. As an example, patients may be administered two doses daily at approximately 12-hour intervals. In some embodiments, the agent is administered once a day.

The agent(s) may be administered on a routine schedule. As used herein a routine schedule refers to a predetermined designated period of time. The routine schedule may encompass periods of time which are identical, or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the invention provides that the agent(s) may be taken orally and that the timing of which is or is not dependent upon food intake. Thus, for example, the agent can be taken every morning and/or every evening, regardless of when the patient has eaten or will eat.

### III. DEFINITIONS

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanyl” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “thiocarbonyl” means —C(═S)—; “sulfonyl” means —S(O)₂—; and “sulfinyl” means —S(O)—.

In the context of chemical formulas, the symbol “—” means a single bond, “═” means a double bond, and “≡” means triple bond. The symbol “ [symbol] ” represents an optional bond, which if present is either single or double. The symbol “ [symbol] ” represents a single bond or a double bond. Thus, the formula

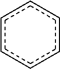

covers, for example,

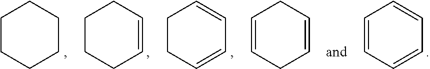

And it is understood that no one such ring atom forms part of more than one double bond. Furthermore, it is noted that the covalent bond symbol “-”, when connecting one or two stereogenic atoms, does not indicate any preferred stereochemistry. Instead, it covers all stereoisomers as well as mixtures thereof. The symbol [symbol] when drawn perpendicularly across a bond (e.g.,

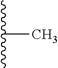

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefine Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a variable is depicted as a “floating group” on a ring system, for example, the group “R” in the formula:

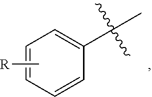

then the variable may replace any hydrogen atom attached to any of the ring atoms, including a depicted, implied, or expressly defined hydrogen, so long as a stable structure is formed. When a variable is depicted as a “floating group” on a fused ring system, as for example the group “R” in the formula:

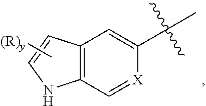

then the variable may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the R enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” or “C=n” defines the exact number (n) of carbon atoms in the group/class. “C≤n” defines the maximum number (n) of carbon atoms that can be in the group/class, with the minimum number as small as possible for the group/class in question. For example, it is understood that the minimum number of carbon atoms in the groups “alkyl_{(C≤8)}”, “cycloalkanediyl_{(C≤8)}”, “heteroaryl_{(C≤8)}”, and “acyl_{(C≤8)}” is one, the minimum number of carbon atoms in the groups “alkenyl_{(C≤8)}”, “alkynyl_{(C≤8)}”, and “heterocycloalkyl_{(C≤8)}” is two, the minimum number of carbon atoms in the group “cycloalkyl_{(C≤8)}” is three, and the minimum number of carbon atoms in the groups “aryl_{(C≤8)}” and “arenediyl_{(C≤8)}” is six. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C5 olefin”, “C5-olefin”, “olefin(_{C5})”, and “olefinc₅” are all synonymous. Except as noted below, every carbon atom is counted to determine whether the group or compound falls with the specified number of carbon atoms. For example, the group dihexylamino is an example of a dialkylamino_{(C=12)} group; however, it is not an example of a dialkylamino_{(C=6)} group. Likewise, phenylethyl is an example of an aralkyl_{(C=8)} group. When any of the chemical groups or compound classes defined herein is modified by the term “substituted”, any carbon atom in the moiety replacing the hydrogen atom is not counted. Thus methoxyhexyl, which has a total of seven carbon atoms, is an example of a substituted alkyl (C1-6). Unless specified otherwise, any chemical group or compound class listed in a claim set without a carbon atom limit has a carbon atom limit of less than or equal to twelve.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” signifies that the compound or chemical group so modified is an acyclic or cyclic, but non-aromatic compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” signifies that the compound or chemical group so modified has a planar unsaturated ring of atoms with 4n+2 electrons in a fully conjugated cyclic π system. An aromatic compound or chemical group may be depicted as a single resonance structure; however, depiction of one resonance structure is taken to also refer to any other resonance structure. For example:

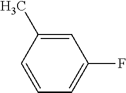

is also taken to refer to

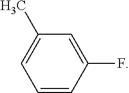

Aromatic compounds may also be depicted using a circle to represent the delocalized nature of the electrons in the fully conjugated cyclic a system, two non-limiting examples of which are shown below;

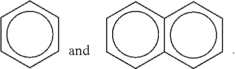

The term “alkyl” refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂(i-Pr, ⁱPr or isopropyl), —CH₂CH₂CH₂CH₃ (n-Bu), —CH(CH₃)CH₂CH₃ (sec-butyl), —CH₂CH(CH₃)₂(isobutyl), —C(CH₃)₃(tert-butyl, t-butyl, t-Bu or ^{t}Bu), and —CH₂C(CH₃)₃(neo-pentyl) are non-limiting examples of alkyl groups. The term “alkanediyl” refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. The term “alkylidene” refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the class of compounds having the formula H-R, wherein R is alkyl as this term is defined above.

The term “cycloalkyl” refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂(cyclopropyl), cyclobutyl, cyclopentyl, or cyclohexyl (Cy). As used herein, the term does not preclude the presence of one or more alkyl groups (carbon number limitation permitting) attached to a carbon atom of the non-aromatic ring structure. The term “cycloalkanediyl” refers to a divalent saturated aliphatic group with two carbon atoms as points of attachment, no carbon-carbon double or triple bonds, and no atoms other than carbon and hydrogen. The group

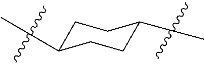 [SMILES: *C1CCC(*)CC1]

is a non-limiting example of cycloalkanediyl group. A “cycloalkane” refers to the class of compounds having the formula H-R, wherein R is cycloalkyl as this term is defined above.

The term “alkenyl” refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” refers to a divalent unsaturated aliphatic group, with two carbon atoms as points of attachment, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H-R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule.

The term “aryl” refers to a monovalent unsaturated aromatic group with an aromatic carbon atom as the point of attachment, said carbon atom forming part of a one or more aromatic ring structures, each with six ring atoms that are all carbon, and wherein the group consists of no atoms other than carbon and hydrogen. If more than one ring is present, the rings may be fused or unfused. Unfused rings are connected with a covalent bond. As used herein, the term aryl does not preclude the presence of one or more alkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl (e.g., 4-phenylphenyl). The term “arenediyl” refers to a divalent aromatic group with two aromatic carbon atoms as points of attachment, said carbon atoms forming part of one or more six-membered aromatic ring structures, each with six ring atoms that are all carbon, and wherein the divalent group consists of no atoms other than carbon and hydrogen. As used herein, the term arenediyl does not preclude the presence of one or more alkyl groups (carbon number limitation permitting) attached to the first aromatic ring or any additional aromatic ring present. If more than one ring is present, the rings may be fused or unfused. Unfused rings are connected with a covalent bond. Non-limiting examples of arenediyl groups include:

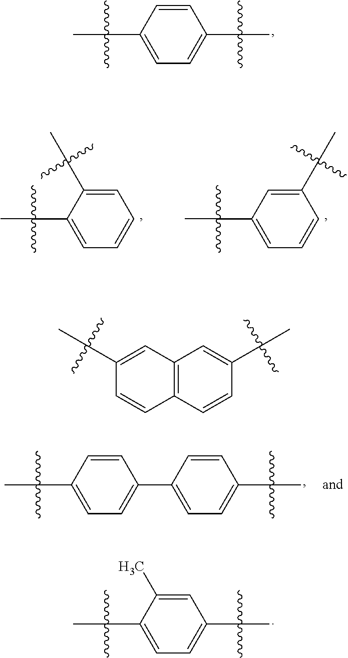

An “arene” refers to the class of compounds having the formula H-R, wherein R is aryl as that term is defined above. Benzene and toluene are non-limiting examples of arenes.

The term “heteroaryl” refers to a monovalent aromatic group with an aromatic carbon atom or nitrogen atom as the point of attachment, said carbon atom or nitrogen atom forming part of one or more aromatic ring structures, each with three to eight ring atoms, wherein at least one of the ring atoms of the aromatic ring structure(s) is nitrogen, oxygen or sulfur, and wherein the heteroaryl group consists of no atoms other than carbon, hydrogen, aromatic nitrogen, aromatic oxygen and aromatic sulfur. If more than one ring is present, the rings are fused; however, the term heteroaryl does not preclude the presence of one or more alkyl or aryl groups (carbon number limitation permitting) attached to one or more ring atoms. Non-limiting examples of heteroaryl groups include benzoxazolyl, benzimidazolyl, furanyl, imidazolyl (Im), indolyl, indazolyl (Im), isoxazolyl, methylpyridinyl, oxazolyl, oxadiazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. A “heteroarene” refers to the class of compounds having the formula H-R, wherein R is heteroaryl. Pyridine and quinoline are non-limiting examples of heteroarenes.

The term “heterocycloalkyl” refers to a monovalent non-aromatic group with a carbon atom or nitrogen atom as the point of attachment, said carbon atom or nitrogen atom forming part of one or more non-aromatic ring structures, each with three to eight ring atoms, wherein at least one of the ring atoms of the non-aromatic ring structure(s) is nitrogen, oxygen or sulfur, and wherein the heterocycloalkyl group consists of no atoms other than carbon, hydrogen, nitrogen, oxygen and sulfur. If more than one ring is present, the rings may be fused, bridged, or spirocyclic. As used herein, the term does not preclude the presence of one or more alkyl groups (carbon number limitation permitting) attached to one or more ring atoms. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, tetrahydropyridinyl, pyranyl, oxiranyl, and oxetanyl. The term “N-heterocycloalkyl” refers to a heterocycloalkyl group with a nitrogen atom as the point of attachment. N-pyrrolidinyl is an example of such a group.

The term “acyl” refers to the group —C(O)R, in which R is a hydrogen, alkyl, cycloalkyl, or aryl as those terms are defined above. The groups, —CHO, —C(O)CH₃ (acetyl, Ac), —C(O)CH₂CH₃, —C(O)CH(CH₃)₂, —C(O)CH(CH₂)₂, —C(O)C₆H₅, and —C(O)C₆H₄CH₃ are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkyl group, as defined above, attached to a —CHO group.

The term “alkoxy” refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂(isopropoxy), or —OC(CH₃)₃(tert-butoxy). The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkylthio” and “acylthio” refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group.

The term “alkylamino” refers to the group —NHR, in which R is an alkyl, as that term is defined above. Non-limiting examples include: —NHCH₃ and —NHCH₂CH₃. The term “dialkylamino” refers to the group —NRR′, in which R and R′ can be the same or different alkyl groups. Non-limiting examples of dialkylamino groups include: —N(CH₃)₂ and —N(CH₃)(CH₂CH₃). The term “amido” (acylamino), when used without the “substituted” modifier, refers to the group —NHR, in which R is acyl, as that term is defined above. A non-limiting example of an amido group is —NHC(O)CH₃.

When a chemical group is used with the “substituted” modifier, one or more hydrogen atom has been replaced, independently at each instance, by —OH, —F, —Cl, —Br, —I, —NH₂, —NO₂, —CO₂H, —CO₂CH₃, —CO₂CH₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. For example, the following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups. Non-limiting examples of substituted aralkyls are: (3-chlorophenyl)-methyl, and 2-chloro-2-phenyl-eth-1-yl. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups. The groups —NHC(O)OCH₃ and —NHC(O)NHCH₃ are non-limiting examples of substituted amido groups.

The use of the word “a” or “an,” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

Throughout this application, the term “about” or “approximately” is used to indicate that a value includes the inherent variation of error for the device, the method being employed to determine the value, or the variation that exists among the study subjects or patients. The term “about” or “approximately”, where used herein when referring to a measurable value such as an amount, a temporal duration, and the like, is meant to encompass, for example, variations of ±20% or ±10%, or ±5%, or ±10%, or ±0.10% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 80% of the time, at least 90% of the time, at least 91% of the time, at least 92% of the time, at least 93% of the time, at least 94% of the time, at least 95% of the time, at least 96% of the time, at least 97% of the time, at least 98% of the time, or at least 99% of the time

An “active ingredient” (AI) or active pharmaceutical ingredient (API) (also referred to as an active compound, active substance, active agent, pharmaceutical agent, agent, biologically active molecule, or a therapeutic compound) is the ingredient in a pharmaceutical drug that is biologically active.

The term “ameliorate” means a detectable or measurable improvement in a subject's condition, disease or symptom thereof. A detectable or measurable improvement includes a subjective or objective decrease, reduction, inhibition, suppression, limit or control in the occurrence, frequency, severity, progression, or duration of the condition or disease, or an improvement in a symptom or an underlying cause or a consequence of the disease, or a reversal of the disease. A successful treatment outcome can lead to a “therapeutic effect,” or “benefit” of ameliorating, decreasing, reducing, inhibiting, suppressing, limiting, controlling or preventing the occurrence, frequency, severity, progression, or duration of a disease or condition, or consequences of the disease or condition in a subject.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to the patient or subject, is sufficient to effect such treatment or prevention of the disease as those terms are defined below.

As used herein any reference to “one embodiment” or “an embodiment” means that a particular element, feature, composition, structure, or characteristic described in connection with the embodiment is included in at least one embodiment. The appearances of the phrase “in one embodiment” in various places in the specification are not necessarily all referring to the same embodiment.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of antiadherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, horse, sheep, goat, dog, cat, mouse, rat, guinea pig, or transgenic species thereof. In certain embodiments, the patient or subject is a primate. Non-limiting examples of human patients are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include: liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

A “pharmaceutical drug” (also referred to as a pharmaceutical, pharmaceutical preparation, pharmaceutical composition, pharmaceutical formulation, pharmaceutical product, medicinal product, medicine, medication, medicament, or simply a drug, agent, or preparation) is a composition used to diagnose, cure, treat, or prevent disease, which comprises an active pharmaceutical ingredient (API) (defined above) and optionally contains one or more inactive ingredients, which are also referred to as excipients (defined above).

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prodrug” means a compound that is convertible in vivo metabolically into an active pharmaceutical ingredient of the present invention. The prodrug itself may or may not have activity with in its prodrug form. For example, a compound comprising a hydroxy group may be administered as an ester that is converted by hydrolysis in vivo to the hydroxy compound. Non-limiting examples of suitable esters that may be converted in vivo into hydroxy compounds include acetates, citrates, lactates, phosphates, tartrates, malonates, oxalates, salicylates, propionates, succinates, fumarates, maleates, methylene-bis-p-hydroxynaphthoate, gentisates, isethionates, di-p-toluoyltartrates, methanesulfonates, ethanesulfonates, benzenesulfonates, p-toluenesulfonates, cyclohexylsulfamates, quinates, and esters of amino acids. Similarly, a compound comprising an amine group may be administered as an amide that is converted by hydrolysis in vivo to the amine compound.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease or symptom thereof in a subject or patient that is experiencing or displaying the pathology or symptomatology of the disease.

The term “unit dose” refers to a formulation of the compound or composition such that the formulation is prepared in a manner sufficient to provide a single therapeutically effective dose of the active ingredient to a patient in a single administration. Such unit dose formulations that may be used include but are not limited to a single tablet, capsule, or other oral formulations, or a single vial with a syringeable liquid or other injectable formulations.

A decrease or reduction in worsening, such as stabilizing the condition or disease, is also a successful treatment outcome. A therapeutic benefit therefore need not be complete ablation or reversal of the disease or condition, or any one, most or all adverse symptoms, complications, consequences or underlying causes associated with the disease or condition. Thus, a satisfactory endpoint may be achieved when there is an incremental improvement such as a partial decrease, reduction, inhibition, suppression, limit, control, or prevention in the occurrence, frequency, severity, progression, or duration, or inhibition or reversal of the condition or disease (e.g., stabilizing), over a short or long duration of time (hours, days, weeks, months, etc.). Effectiveness of a method or use, such as a treatment that provides a potential therapeutic benefit or improvement of a condition or disease, can be ascertained by various methods and testing assays.

The above definitions supersede any conflicting definition in any reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### IV. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Molecular Dynamics Simulations

The orientation of the bc₁ complex (PDB: 1NTZ) with respect to the membrane was computed using the Peripheral Proteins in Membranes (PPM) web server (Lomize et al., 2012). The oriented protein was inserted in the membrane using the CHARMM-GUI Membrane Builder (Jo et al., 2008) online tool using a membrane composition of Phosphatidylcholine (PC): Phosphatidylethanolamine (PE): Cardiolipin (CL) equal to 50:30:20 (Zinser et al., 1991). The protein-membrane system was placed in a water box with 0.15M KCl neutralizing ions. The assembled system was energy minimized and then equilibrated using the GROMACS (Lindahl et al., 2001) program with CHARMM PARAM36 force field (Brooks et al., Best et al., 2012) under periodic boundary conditions. Afterward, molecular dynamics (MD) simulation was performed on the assembled system for 1000 ns. MD simulations for the bc₁ complex with pharmacophores bound at the NQ binding site were performed as mentioned above, starting with the bc₁ complex with the most stable docked conformation of the pharmacophores at the NQ binding site.

Virtual screening calculation: 1,489,806 compounds from the ZINC20 database were selected (Irwin et al., 2020) with a range of molecular weight of 250-550 Daltons and Log P of 0-5, satisfying the druglike properties based on Lipinski's Rule of 5. The virtual screening simulations were performed using the PyRx package (Dallakyan and Olson, 2015) with the Autodock Vina (Eberhardt et al., 2021) scoring method and at multiple stages: First, virtual screening of 1,489,806 compounds against the NQ site were performed where its residues were allowed to be flexible and using exhaustiveness equals 12 and number of modes equals 10. 10,038 compounds that bind at the NQ site more strongly than the native ligand UQH₂ were then selected. Next, a virtual screening of the 10,038 compounds against the NQ site with all its residues fixed was performed. The 4,751 compounds that bind strongly at the NQ site in the flexible mode by at least 5 kcal/mol compared to the fixed mode were then selected. Then, a virtual screening of the 4,751 compounds against the Qₒ site was performed with its residues being flexible. The 540 compounds that bind at the Qₒ higher than UQH₂ were then selected. Finally, a virtual screening of the 540 compounds against the Qᵢ site with its flexible residues, was performed yielding 272 compounds that bind at the Qᵢ site higher than UQH₂.

### Example 2—Total Cellular ROS and Mitochondrial Superoxide Anion Measurement Assay

The MCF7 breast cancer cell line (ATCC, HTB-22) was purchased from American Type Culture Collection (ATCC, Manassas, VA) and propagated in Eagle's minimum essential medium (EMEM, ATCC) supplemented with 10% FBS (Thermo Scientific), 1% L-glutamine and 1% penicillin/streptomycin (ATCC) in a humidified incubator at 37° C. and 5% CO₂. Cells were harvested and seeded into a 96-well black plate at a cell density of 25,000 cells/well and left overnight to adhere. Afterward, cells were treated with 25 μM of the 14 purchased ligands from MolPort (Riga, Latvia), positive control (i.e., doxorubicin), and negative control (i.e., DMSO) and left in the incubator for 6-8 hours. For the Qₒ and Qᵢ inhibition experiments, 25 μM of myxothiazol or antimycin A (Sigma-Aldrich), respectively were also incubated. Negative controls for the inhibition experiments were composed of DMSO and either 25 μM of myxothiazol or antimycin A. Afterward, cells were washed and stained for 30 minutes at 37° C. and 5% CO₂ in the dark using the CellRox Green (Invitrogen, USA) or the MitoSOX Red Assay Kit (Invitrogen, USA). Afterward, cells were washed, and total cellular ROS or mitochondrial superoxide anion levels were measured using the Synergy Biotek UV/Vis/fluorescence microplate reader with excitation/emission wavelengths of 490/525 nm or 396/610 nm, respectively. Nuclei were labeled using NucBlue Live ReadyProbes Reagent (Hoechst 33342). All experiments were performed in at least triplicate and repeated for at least three independent trials.

### Example 3—MTS Assay

MCF7 cells were harvested and seeded at a cell density of 5,000 cells/well into a 96-well white plate and left overnight to adhere. Afterward, cells were treated with different concentrations (0, 5, 10, 25, 50, and 100 μM) of pharmacophores, positive control (i.e., tamoxifen and doxorubicin), and negative control (i.e., DMSO) for 48 hours. Then, cells were washed and assayed for cytotoxicity using CellTiter 96® AQueous One Solution Cell Proliferation Assay kit (MTS) (Promega, USA), which is a colorimetric assay containing colorless tetrazolium compound that is bio-reduced by NADPH or NADH produced by dehydrogenase enzymes in metabolically active cells into a colored formazan product that can be measured at wavelength 490 nm using the Synergy Biotek plate reader. Sequential treatment was performed by incubating the overnight attached MCF7 cells with 25 μM of MH-200102 for 24 hours. Then, the cells were washed once and incubated with different concentrations of MH-101015 for another 24 hours. Then, the cells were assayed for cytotoxicity, as mentioned above.

### Example 4—Results and Analysis

Statistical Analysis: All the data and results obtained by three independent experiments are expressed as the mean fold change compared to negative control±SD. Comparisons between groups were determined using Student's t-test. A difference with P<0.05 was considered to be significant.

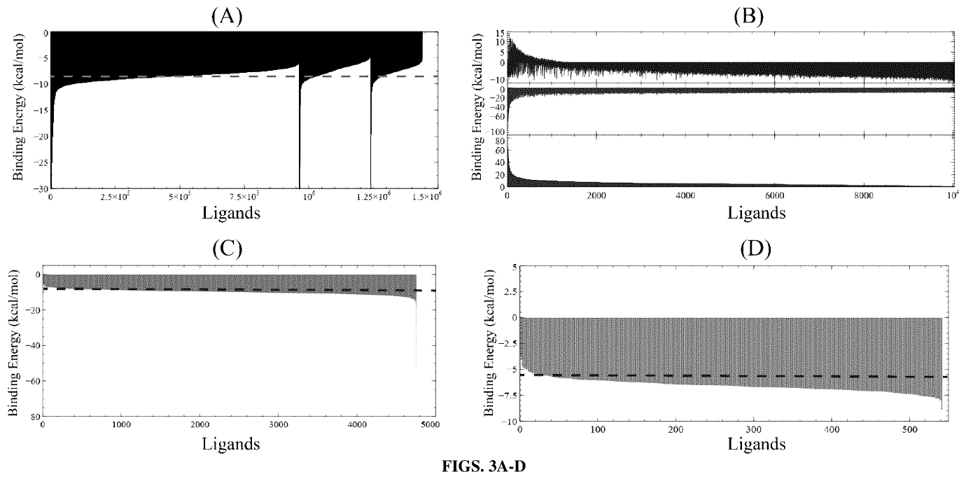

A system composed of the bc₁ complex (PDB: 1NTZ) embedded in a membrane with a 50:30:20 composition of PC:PE:CL was initially constructed, surrounded by a water box and simulated for 1000 ns under periodic boundary conditions to obtain a more realistic structure of the bc₁ complex. Afterward, virtual screening of 1,489,806 compounds from the ZINC20 database at multiple stages was performed to ensure that the obtained set of compounds has the following properties as follows: 1—Screening of 1,489,806 compounds and the native ligands (UQ and UQH₂) against the NQ site and selecting 10,038 pharmacophores that bind more strongly at the NQ site compared to the native ligands and hence suffer no competition upon binding at the NQ site (FIG. 3A). 2—screening of the selected 10,038 compounds against the NQ site in both flexible mode (i.e., the residues of the NQ-site are allowed to move) and fixed mode (i.e., the residues of the NQ-site are fixed in position) and selecting 4,751 compounds that bind strongly in flexible mode by at least 5 kcal/mol compared to the fixed mode and hence would potentially induce conformational changes at the NQ site that could eventually modulate the conformation of the key phenylalanine residue (Phe90) (FIG. 3B). 3—screening of 4,751 compounds against the Qₒ site (FIG. 3C) and Qᵢ site (FIG. 3D) to eventually obtain 272 compounds that bind preferentially at the NQ site compared to UQ and UQH₂ but less firmly at the Qₒ and the Qᵢ sites (Hagras, 2024).

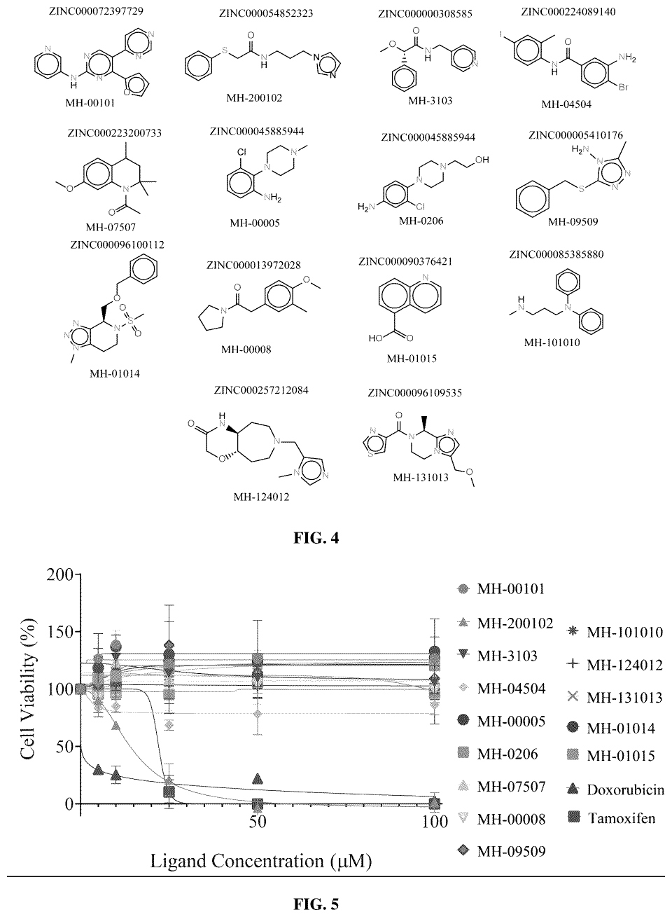

The top 14 ligands out of the obtained 272 compounds that were commercially available through Molport were then picked (FIG. 4). The biological activities of those 14 ligands with respect to their cytotoxic and ROS-modulating activities were then screened. A cytotoxicity assay for all the purchased 14 pharmacophores was performed with tamoxifen and doxorubicin as the positive controls and DMSO as the negative control against the MCF7 cell line. It was found that the MH-200102 compound has a cytotoxic activity with IC₅₀ equal to 14.57 μM, lower than IC₅₀ of tamoxifen (˜22.07 μM) (Seeger et al., 2004) yet higher than the IC₅₀ of doxorubicin (˜1 μM) (Fang et al., 2014) (FIG. 5). Additionally, MH-04504 showed a modest cytotoxic activity compared to that of MH-200102. Other compounds showed either proliferative activity or no activity at all.

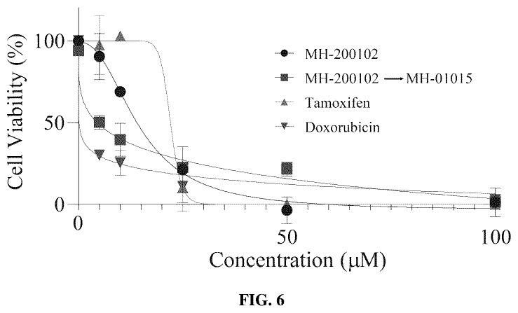

More interestingly, it was found that sequential treatment with MH-200102→MH-01015 showed higher cytotoxic activity than the single treatment with MH-200102 (FIG. 6). It was found that the sequential treatment with MH-200102 and then MH-01015 lowered the IC₅₀ to 5 μM compared to 14.57 μM with a single treatment with MH-200102 (FIG. 6).

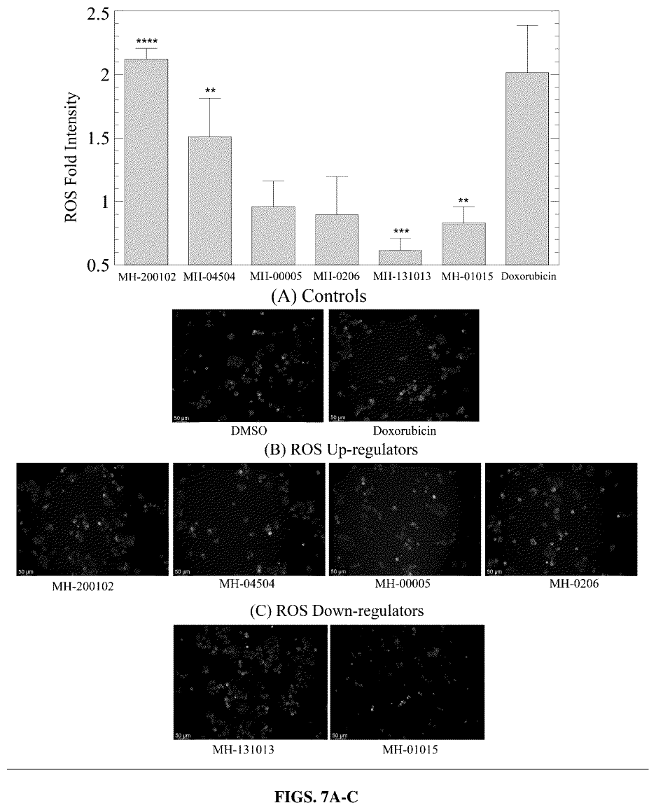

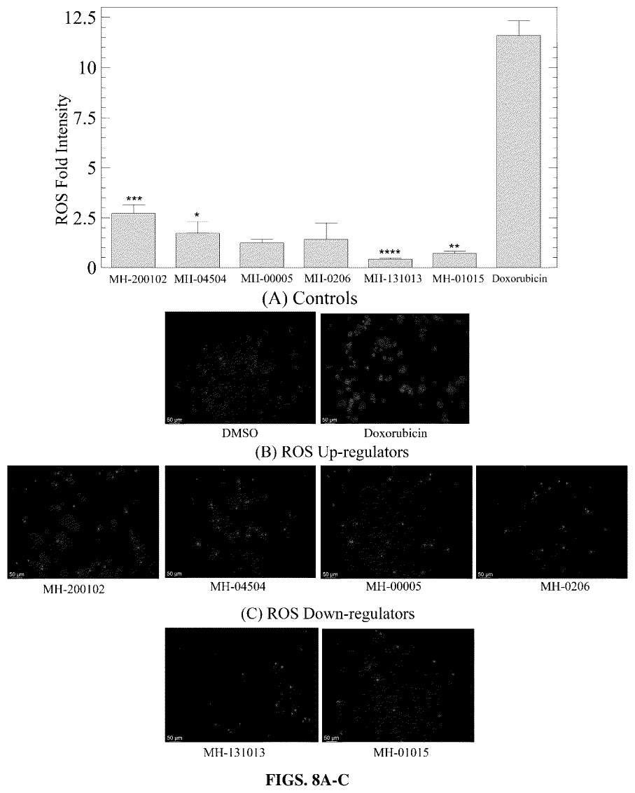

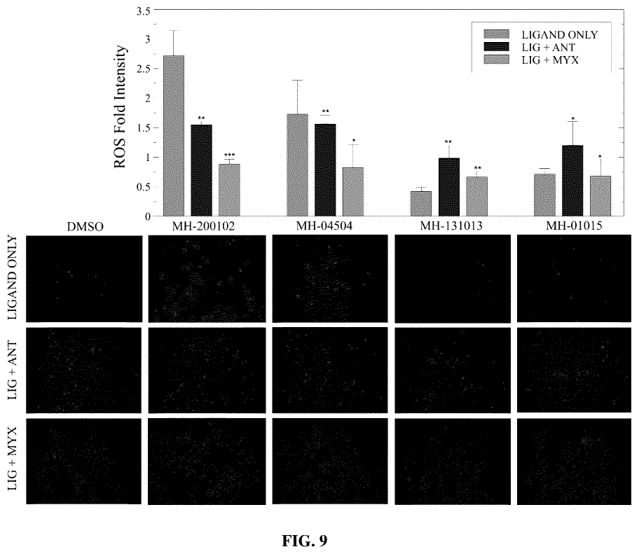

The biochemical assays for the total cellular ROS levels showed that the MH-200102 exhibits an almost twofold increase in the total ROS upregulating activity compared to the ROS level of the negative control (i.e., DMSO) against the MCF7 cells, while the MH-04504 shows a modest total ROS upregulating activity (FIG. 7). More interestingly, it was found that MH-131013 and MH-01015 show total ROS down-regulating activity compared to the ROS level of the negative control (FIG. 7). Additionally, the mitochondrial superoxide radical anion levels were measured where it was found that the two ROS up-regulator compounds (MH-200102 and MH-04504) and the two ROS down-regulator compounds (MH-131013 and MH-01015) show a similar regulating activity on the mitochondrial superoxide anion levels proportional to their regulatory activities for the total cellular ROS levels (FIG. 8).

To verify if the discovered ROS-regulator compounds bind at the NQ site of the bc₁ complex, the mitochondrial superoxide anion levels were additionally measured for each one of the lead-hit ROS regulators with either myxothiazole (MYX) or antimycin. MYX is a specific inhibitor for the Qₒ site of the bc₁ complex and hence blocks its catalytic activity (Von Jagow et al., 1986). For the ROS up-regulators, the results show a decrease in the mitochondrial superoxide anion levels in the MCF7 cells treated with both ligand and MYX compared to ligand only (FIG. 9). Those results show that MYX blocks the ROS up-modulating activity of MH-200102 and MH-04504. For the ROS down-regulators, it was found that treatment with both ligand and MYX restores the mitochondrial superoxide levels to that of the negative control (i.e., DMSO and MYX). Hence, those results also show that MYX blocks any ROS down-modulating activity of MH-131013 and MH-01015.

Antimycin (ANT) is a specific inhibitor for the Qᵢ site of the bc₁ complex and hence enhances the reverse electron transfer reaction in the bc₁ complex, increasing the mitochondrial superoxide anion levels (Quinlan et al., 2011; Sun and Trumpower, 2003). It was found that treatment with ROS up-regulator and ANT has a higher superoxide anion level than negative control treatment (i.e., DMSO and ANT). Those results show a synergistic activity between the ROS up-regulator and ANT in elevating the mitochondrial superoxide anion levels. On the other hand, treatment with both ROS down-regulator and ANT shows a comparable mitochondrial superoxide anion level to the negative control treatment, which emphasizes that ANT blocks any ROS down-regulating activity of the ligands MH-131013 and MH-01015.

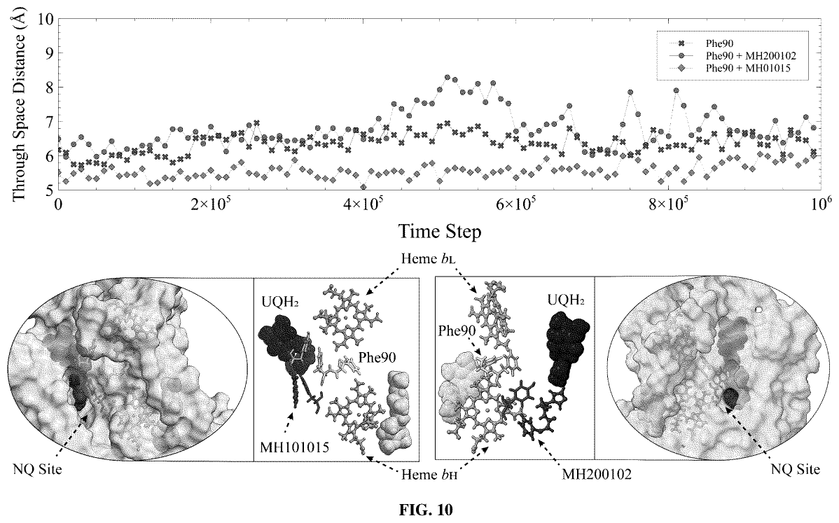

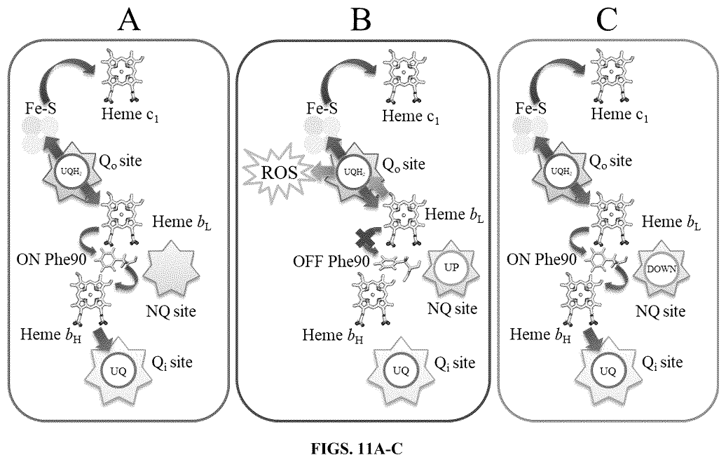

To understand the molecular activities of the ROS up-regulators and down-regulators, the molecular dynamics of the bc₁ complex were simulated where one monomer has UQH₂, UQ, and either MH-200102 or MH-01015 ligands docked at the Qₒ, Qᵢ, and NQ sites, respectively while leaving the other monomer ligand-free. The electron transfer (ET) was measured through-space distance between the heme b_{L}, Phe90, and heme b_{H} for the ligand-free monomer and for both MH-200102 or MH-01015 docked monomers (FIG. 10). The results show that the ROS up-regulator MH-200102 increases ET through-space distance to ˜6.5 Å in close agreement with the ligand-free monomer. On the other hand, MH-01015 reduces ET through-space distance to ˜5.5 Å, as observed previously (Hagras, 2024). Interestingly, it was found that the MH-200102 interacts unfavorably with Tyr95 residue where their aromatic rings stack sideways and hence destabilizing Tyr95, leading to a series of aromatic-aromatic interactions that eventually orient Phe90 in unfavorable conformation for ET between heme b_{L} and heme b_{H} (i.e., OFF conformation as previously described (Hagras, 2024; Hagras et al., 2015; Hagras and Stuchebrukhow, 2021; Hagras and Stuchebrukhow, 2016) (FIG. 10, right inset). On the other hand, it was found that the MH-01015 interacts favorably with Tyr95 through face-to-face configuration (Anjana et al., 2012; Burley and Petsko, 1985) that was found to eventually induce favorable conformation of Phe90 (i.e., ON conformation as previously described (Hagras, 2024; Hagras et al., 2015; Hagras and Stuchebrukhow, 2021; Hagras and Stuchebrukhow, 2016)) (FIG. 10, left inset).

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFFERENCES

The following references, such that they provide exemplary procedural or other details supplementary to those set forth herein, are specifically incorporated by reference.
U.S. Pat. No. 11,058,645
Anderson, Practical Process Research & Development—A Guide for Organic Chemists, 2nd ed., Academic Press, New York, 2012.
Abbas et al., “The role of reactive oxygen species in immunopathogenesis of rheumatoid arthritis,” Iranian Journal of Allergy, Asthma and Immunology, 195-202, 2008.
Ahlemeyer and Krieglstein, “Retinoic acid reduces staurosporine-induced apoptotic damage in chick embryonic neurons by suppressing reactive oxygen species production.”
Anjana et al., “Aromatic-aromatic interactions in structures of proteins and protein-DNA complexes: a study based on orientation and distance,” Bioinformation, 8:1220, 2012.
Best et al., “Optimization of the additive CHARMM all-atom protein force field targeting improved sampling of the backbone phi, psi and side-chain chi(1) and chi(2) dihedral angles,” Journal of chemical theory and computation, 8:3257-3273, 2012.
Bolduc et al., “Reactive oxygen species, aging and articular cartilage homeostasis,” Free Radical Biol. Med., 132:73-82, 2019.
Boonstra and Post, “Molecular events associated with reactive oxygen species and cell cycle progression in mammalian cells,” Gene, 337:1-13, 2004.
Brandt and Trumpower, “The Protonmotive Q Cycle in Mitochondria and Bacteria, Crit. Rev.” Biochem. Mol. Biol., 29:165-197, 1994.
Brooks et al., “CHARMM: the biomolecular simulation program,” J. Comput. Chem., 30:1545-1614, 2009.
Burley and Petsko, “Aromatic-Aromatic Interaction: A Mechanism of Protein Structure Stabilization,” Science, 229:23-28, 1985.
Chang et al., “Reactive oxygen species production is involved in quercetin-induced apoptosis in human hepatoma cells.”
Crofts et al., “Mechanism of Ubiquinol Oxidation by the be 1 Complex: Role of the Iron Sulfur Protein and Its Mobility,” Biochemistry, 38:15791-15806, 1999.
Crofts et al., “Pathways for proton release during ubihydroquinone oxidation by the bc(1) complex,” Proc. Natl. Acad. Sci. U.S.A., 96:10021-10026, 1999.
Crofts et al., “The modified Q-cycle explains the apparent mismatch between the kinetics of reduction of cytochromes c1 and b_{H} in the bc1 complex,” J. Biol. Chem., 278:36191-36201, 2003.
Dallakyan and Olson, “Small-molecule library screening by docking with PyRx, Methods in molecular biology” Clifton, N.J., 1263:243-250, 2015.
Drose and Brandt, “The mechanism of mitochondrial superoxide production by the cytochrome bc1 complex,” J. Biol. Chem., 283:21649-21654, 2008.
Eberhardt et al., “S.J.J.o.c.i. Forli, modeling, AutoDock Vina 1.2. 0: New docking methods, expanded force field, and python bindings,” 61:3891-3898, 2021.
Fang et al., “Doxorubicin induces drug resistance and expression of the novel CD44st via NF-κB in human breast cancer MCF-7 cells,” Oncol. Rep., 31:2735-2742, 2014.
Gong et al., “Rutin inhibits hydrogen peroxide-induced apoptosis through regulating reactive oxygen species mediated mitochondrial dysfunction pathway in human umbilical vein endothelial cells.”
Hagras and Stuchebrukhov, “Inhibition of respiratory complex III by ligands that interact with a regulatory switch,” U.S. Pat. No. 11,058,645:2021, 2021.
Hagras and Stuchebrukhov, “Internal switches modulating electron tunneling currents in respiratory complex III,” Biochimica et Biophysica Acta (BBA)-Bioenergetics, 1857:749-758, 2016.
Hagras and Stuchebrukhov, “Novel inhibitors for a novel binding site in respiratory complex III,” The Journal of Physical Chemistry B, 120:2701-2708, 2016.
Hagras et al., “Quantum calculations of electron tunneling in respiratory complex III,” The Journal of Physical Chemistry B, 119:14637-14651, 2015.
Hagras, “Respiratory Complex III: A Bioengine with a Ligand-Triggered Electron-Tunneling Gating Mechanism,” The Journal of Physical Chemistry B, 128:990-1000, 2024.
Hagras, “Respiratory Complex III: A Bioengine with a Ligand-Triggered Electron-Tunneling Gating Mechanism,” The Journal of Physical Chemistry B, 2024.
Ham et al., “Spirafolide from bay leaf (Laurus nobilis) prevents dopamine-induced apoptosis by decreasing reactive oxygen species production in human neuroblastoma SH-SY5Y cells.”
Handbook of Pharmaceutical Salts: Properties, and Use, Stahl and Wermuth Eds., Verlag Helvetica Chimica Acta, 2002.
Hogan and Wheeler, “The complex roles of NADPH oxidases in fungal infection”, Cellular Microbiology, 16(8):1156-1167, 2014.
Irwin et al., “ZINC20—A Free Ultralarge-Scale Chemical Database for Ligand Discovery, Journal of Chemical Information and Modeling,” 60:6065-6073, 2020.
Jo et al., “CHARMM-GUI: A web-based graphical user interface for CHARMM,” J. Comput. Chem., 29:1859-1865, 2008.
Kang et al., “L-ascorbic acid (vitamin C) induces the apoptosis of B16 murine melanoma cells via a caspase-8-independent pathway.”
Kumar et al., “Hydrogen peroxide induced apoptosis in amnion-derived WISH cells is not inhibited by vitamin C.”
Lanciano et al., “Molecular mechanisms of superoxide production by complex III: a bacterial versus human mitochondrial comparative case study,” Biochimica et Biophysica Acta (BBA)-Bioenergetics, 1827:1332-1339, 2013.
Li et al., “Roles of reactive oxygen species in cell signaling pathways and immune responses to viral infections” Arch Virol. 162:603-610, 2017.
Lindahl et al., “GROMACS 3.0: a package for molecular simulation and trajectory analysis,” J. Mol. Model., 7:306-317, 2001.
Lomize et al., “OPM database and PPM web server: resources for positioning of proteins in membranes,” Nucleic Acids Res., 40:D370-376, 2012.
López-Armada et al., “Mitochondrial dysfunction and the inflammatory response,” Mitochondrion, 13:106-118, 2013.
Manoharan et al., “The role of reactive oxygen species in the pathogenesis of Alzheimer's disease, Parkinson's disease, and Huntington's disease: a mini review, Oxidative medicine and cellular longevity,” 2016, 2016.
Marschall and Tudzynski, “Reactive oxygen species in development and infection processes”, Semin. Cell Dev. Biol., 138-146, 2016.
McGarry et al., “Hypoxia, oxidative stress and inflammation,” Free Radical Biol. Med., 125:15-24, 2018.
Mitchell, “Coupling of Phosphorylation to Electron and Hydrogen Transfer by a Chemi-Osmotic type of Mechanism,” Nature, 191:144-148, 1961.
Mitchell, “Possible molecular mechanisms of the protonmotive function of cytochrome systems,” J. Theor. Biol., 62:327-367, 1976.
Mitchell, “The protonmotive Q cycle: a general formulation,” FEBS Lett., 59:137-139, 1975.
Muller, “The nature and mechanism of superoxide production by the electron transport chain: its relevance to aging, Journal of the American Aging Association,” 23:227-253, 2000.
Osyczka, C. C. Moser, P. L. Dutton, Fixing the Q cycle, Trends Biochem. Sci, 30 (2005) 176-182.
Quinlan et al., “The mechanism of superoxide production by the antimycin-inhibited mitochondrial Q-cycle,” J. Biol. Chem., 286:31361-31372, 2011.
Schönfeld and Wojtczak, “Fatty acids decrease mitochondrial generation of reactive oxygen species at the reverse electron transport but increase it at the forward transport,” Biochimica et Biophysica Acta (BBA)-Bioenergetics, 1767:1032-1040, 2007.
Seeger et al., “Inhibition of human breast cancer cell proliferation with estradiol metabolites is as effective as with tamoxifen,” Hormone and metabolic research, 36:277-280, 2004.
Smith, March's Advanced Organic Chemistry: Reactions, Mechanisms, and Structure, 7th Ed., Wiley, 2013.
Sorce et al., “NOX enzymes in the central nervous system: from signaling to disease, Antioxidants & redox signaling,” 11:2481-2504, 2009.
Sun et al., “Superoxide anion generation by the cytochrome bc1 complex,” Arch. Biochem. Biophys., 419:198-206, 2003.
Trachootham et al., “Targeting cancer cells by ROS-mediated mechanisms: a radical therapeutic approach?,” Nature reviews Drug discovery, 8:579-591, 2009.
U ğuz et al., “Curcumin inhibits apoptosis by regulating intracellular calcium release, reactive oxygen species and mitochondrial depolarization levels in SH-SY5Y neuronal cells.”
Von Jagow et al., “Mucidin and strobilurin A are identical and inhibit electron transfer in the cytochrome bc1 complex of the mitochondrial respiratory chain at the same site as myxothiazol,” Biochemistry, 25:775-780, 1986.
Waris and Ahsan, “Reactive oxygen species: role in the development of cancer and various chronic conditions,” Journal of carcinogenesis, 5:14, 2006.
Watson, “Oxidants, antioxidants and the current incurability of metastatic cancers, Open biology,” 3:120144, 2013.
Woo et al., “Molecular mechanisms of curcumin-induced cytotoxicity: induction of apoptosis through generation of reactive oxygen species, down-regulation of Bc1-X L and IAP, the release of cytochrome c and inhibition of Akt,” Carcinogenesis, 24:1199-1208, 2003.
Yang et al., “Mitochondria and mitochondrial ROS in cancer: novel targets for anticancer therapy,” J. Cell. Physiol., 231:2570-2581, 2016.
Yang et al., “Reactive oxygen species in the immune system, International reviews of immunology,” 32:249-270, 2013.
Young et al., “Reactive oxygen species production by the mitochondrial respiratory chain in isolated rat hepatocytes and liver mitochondria: studies using myxothiazol,” Arch. Biochem. Biophys., 405:65-72, 2002.
Zhang et al., “Electron transfer by domain movement in cytochrome bc1,” Nature, 392:677-684, 1998.
Zhang, et al., “ROS and ROS-mediated cellular signaling, Oxidative medicine and cellular longevity,” 2016, 2016.
Zinser et al., “Phospholipid synthesis and lipid composition of subcellular membranes in the unicellular eukaryote Saccharomyces cerevisiae,” J. Bacteriol., 173:2026-2034, 1991.

1. A method of modulating the production of a reactive oxygen species comprising contacting a cell with a compound in an amount sufficient to induce a change in the amount of one or more reactive oxygen species, wherein:
(A) the compound comprises a spirocyclic group;
(B) the compound comprises one or more heteroaromatic groups that contains at least two nitrogen heteroatoms;
(C) the compound comprises one or more sulfonamide groups;
(D) the compound comprises two or more lactams;
(E) the compound comprises one or more heterocycloalkane groups that contain a nitrogen atom in combination with an oxygen atom or a sulfur atom;
(F) the compound comprises one or more piperazine ring;
(G) the compound comprises a fused hetetocycloalkane ring system containing at least two or more ring system one or more nitrogen atoms with at least one seven membered ring;
(H) the compound comprises two or more pendant aromatic ring systems, wherein at least one of the aromatic ring system is a heteroaromatic ring system with at least one nitrogen atom;
(I) the compound comprises two or more fused cycoalkane ring systems, wherein at least one of the cycloalkane rings is a cyclopentane ring, wherein the cycloalkane rings optionally contain one or more heteroatoms;
(J) the compound comprises a triazole group;
(K) the compound is further defined by the formula: 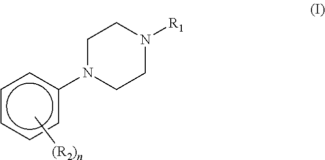
wherein:
R₁ is C1-C12 alkyl or C1-C12 substituted alkyl; and
Each R₂ are each independently selected from amino, halo, hydroxy, C1-C6 alkylamino, substituted C1-C6 alkylamino, C2-C12 dialkylamino, or substituted C2-C12 dialkylamino, or
further defined by the formula: 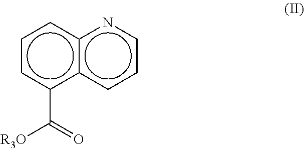
wherein:
R₃ is hydrogen, C1-C8 alkyl or C1-C8 substituted alkyl; or
further defined by the formula: 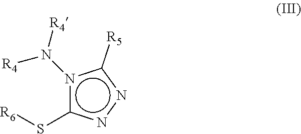
wherein:
R₄, R₄′, and R₅ are each independently selected from hydrogen, C1-C8 alkyl or C1-C8 substituted alkyl; and
R₆ is C7-C12 alkyl or C7-C12 substituted alkyl; or
further defined by the formula: 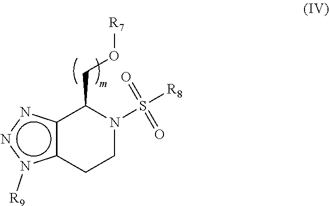
wherein:
m is 1, 2, 3, or 4;
R₇ is C7-C12 alkyl or C7-C12 substituted alkyl; and
R₈ and R₉ are each independently selected from hydrogen, C1-C8 alkyl or C1-C8 substituted alkyl; or
further defined by the formula: 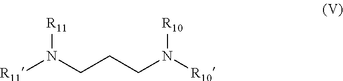
wherein:
R₁₀, R₁₀′, R₁₀, and R₁₀′ are each independently hydrogen, C1-C8 alkyl, C1-C8 substituted alkyl, C6-C12 aryl or C6-C12 substituted aryl; or
further defined by the formula: 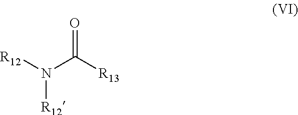
wherein:
R₁₂, R₁₂′, and R₁₃ are each independently hydrogen, C1-C8 alkyl, C1-C8 substituted alkyl, C6-C12 aryl or C6-C12 substituted aryl; or
further defined by the formula: 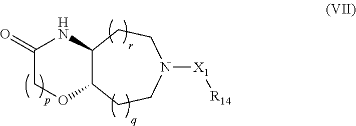
wherein:
p, q, and r are each independently 0, 1, 2, or 3;
X₁ is C1-C6 alkanediyl or C1-C6 substituted alkanediyl; and
R₁₄ is C6-C12 aryl, C6-C12 substituted aryl, C1-C12 heteroaryl or C1-C12 substituted heteroaryl; or
further defined by the formula: 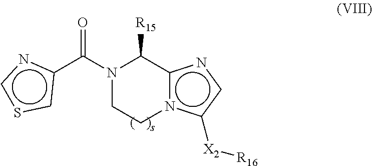
wherein:
s is 0, 1, 2, or 3;
X₂ are C1-C6 alkanediyl or C1-C6 substituted alkanediyl;
R₁₅ is C1-C8 alkyl or C1-C8 substituted alkyl; and
R₁₆ is amino, hydroxy, C1-C12 alkoxy, C1-C12 substituted alkoxy, C1-C12 alkylamino, C1-C12 substituted alkylamino C1-C12 dialkylamino or C1-C12 substituted dialkylamino; or
further defined by the formula: 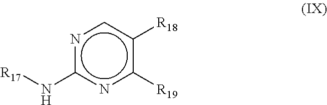
wherein:
R₁₇, R₁₈, and R₁₉ are each independently C1-C12 heteroaryl or C1-C12 substituted heteroaryl; or
further defined by the formula: 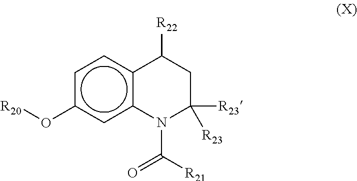
wherein:
R₂₀, R₂₁, R₂₂, R₂₃, and R₂₃′ are each independently C1-C8 alkyl or C1-C8 substituted alkyl; or
further defined by the formula: 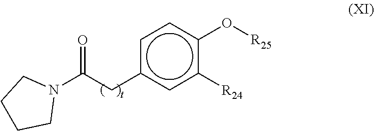
wherein:
t is 1, 2, or 3; and
R₂₄ and R₂₅ are each independently C1-C8 alkyl or C1-C8 substituted alkyl; or
further defined by the formula: 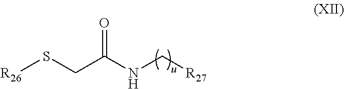
wherein:
u is 1, 2, 3, 4, or 5; and
R₂₆ and R₂₇ are each independently C6-C12 aryl, C6-C12 substituted aryl, C1-C12 heteroaryl or C1-C12 substituted heteroaryl; or
further defined by the formula: 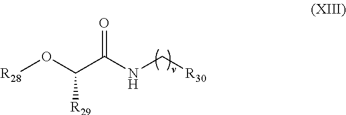
wherein:
v is 1, 2, 3, 4, or 5; and
R₂₈, R₂₉, and R₃₀ are each independently C1-C12 alkyl, C1-C12 substituted alkyl, C6-C12 aryl, C6-C12 substituted aryl, C1-C12 heteroaryl or C1-C12 substituted heteroaryl; and/or
(L) the compound is defined as shown in Table 1.

2. The method of claim 1, wherein the compound is further defined by two or more elements defined in (A)-(K) as described above.

3.-17. (canceled)

18. The method of claim 1, wherein the method results in decreased production of reactive oxygen species.

19. The method of claim 1, wherein the method results in increased production of reactive oxygen species.

20. The method of claim 1, wherein the method results in modulation of one or more reactive oxygen species.

21.-24. (canceled)

25. The method of claim 1, wherein the cell is in vitro.

26. The method of claim 1, wherein the cell is in vivo.

27. The method of claim 1, wherein the cell is in vivo and the method results in the treatment of a disease or disorder.

28. The method of claim 27, wherein the disease or disorder is treated by increasing the production of reactive oxygen species.

29. The method of claim 27, wherein the disease or disorder is treated by decreasing the production of reactive oxygen species.

30. The method of claim 27, wherein the disease or disorder is treated by a combination of pre-treatment with compounds that decrease the production of reactive-oxygen species and then a subsequent treatment with different compounds that increase the production of reactive oxygen species.

31. The method of claim 27, wherein the disease or disorder is treated by a combination of pre-treatment with compounds that increase the production of reactive-oxygen species and then a subsequent treatment with different compounds that decrease the production of reactive oxygen species.

32. A method of treating a disease or disorder in a patient in need thereof comprising administering to the patient a therapeutically effective amount of a reactive oxygen species modulator, wherein the reactive oxygen modulator is further defined as a compound comprising one or more of the following features:
(A) the compound comprises a spirocyclic group;
(B) the compound comprises one or more heteroaromatic groups that contains at least two nitrogen heteroatoms;
(C) the compound comprises one or more sulfonamide groups;
(D) the compound comprises two or more lactams;
(E) the compound comprises one or more heterocycloalkane groups that contain a nitrogen atom in combination with an oxygen atom or a sulfur atom;
(F) the compound comprises one or more piperazine ring;
(G) the compound comprises a fused hetetocycloalkane ring system containing at least two or more ring system one or more nitrogen atoms with at least one seven membered ring;
(H) the compound comprises two or more pendant aromatic ring systems, wherein at least one of the aromatic ring system is a heteroaromatic ring system with at least one nitrogen atom;
(I) the compound comprises two or more fused cycoalkane ring systems, wherein at least one of the cycloalkane rings is a cyclopentane ring, wherein the cycloalkane rings optionally contain one or more heteroatoms;
(J) the compound comprises a triazole group;
(K) the compound is further defined by the formula: 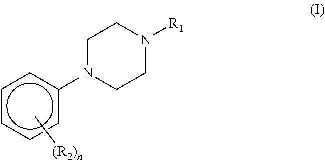
wherein:
R₁ is C1-C12 alkyl or C1-C12 substituted alkyl; and
Each R₂ are each independently selected from amino, halo, hydroxy, C1-C6 alkylamino, substituted C1-C6 alkylamino, C2-C12 dialkylamino, or substituted C2-C12 dialkylamino, or
further defined by the formula: 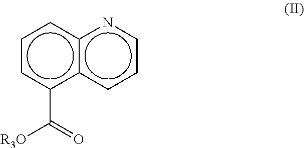
wherein:
R₃ is hydrogen, C1-C8 alkyl or C1-C8 substituted alkyl; or
further defined by the formula: 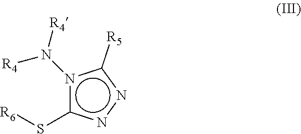
wherein:
R₄, R₄′, and R₅ are each independently selected from hydrogen, C1-C8 alkyl or C1-C8 substituted alkyl; and
R₆ is C7-C12 alkyl or C7-C12 substituted alkyl; or
further defined by the formula: 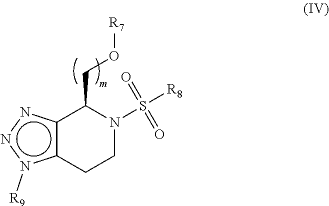
wherein:
m is 1, 2, 3, or 4;
R₇ is C7-C12 alkyl or C7-C12 substituted alkyl; and
R₈ and R₉ are each independently selected from hydrogen, C1-C8 alkyl or C1-C8 substituted alkyl; or
further defined by the formula: 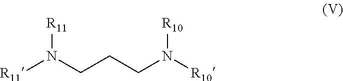
wherein:
R₁₀, R₁₀′, R₁₀, and R₁₀′ are each independently hydrogen, C1-C8 alkyl, C1-C8 substituted alkyl, C6-C12 aryl or C6-C12 substituted aryl; or
further defined by the formula: 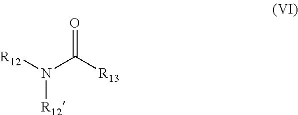
wherein:
R₁₂, R₁₂′, and R₁₃ are each independently hydrogen, C1-C8 alkyl, C1-C8 substituted alkyl, C6-C12 aryl or C6-C12 substituted aryl; or
further defined by the formula: 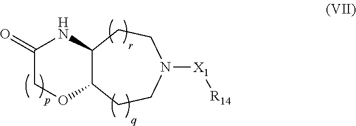
wherein:
p, q, and r are each independently 0, 1, 2, or 3;
X₁ is C1-C6 alkanediyl or C1-C6 substituted alkanediyl; and
R₁₄ is C6-C12 aryl, C6-C12 substituted aryl, C1-C12 heteroaryl or C1-C12 substituted heteroaryl; or
further defined by the formula: 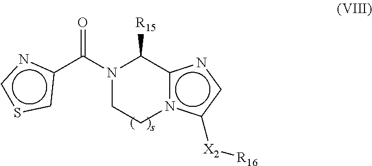
wherein:
s is 0, 1, 2, or 3;
X₂ are C1-C6 alkanediyl or C1-C6 substituted alkanediyl;
R₁₅ is C1-C8 alkyl or C1-C8 substituted alkyl; and
R₁₆ is amino, hydroxy, C1-C12 alkoxy, C1-C12 substituted alkoxy, C1-C12 alkylamino, C1-C12 substituted alkylamino C1-C12 dialkylamino or C1-C12 substituted dialkylamino; or
further defined by the formula: 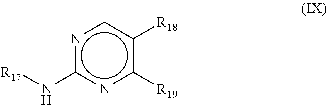
wherein:
R₁₇, R₁₈, and R₁₉ are each independently C1-C12 heteroaryl or C1-C12 substituted heteroaryl; or
further defined by the formula: 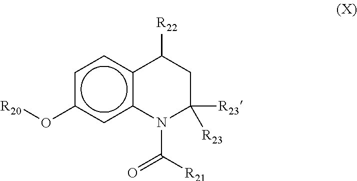
wherein:
R₂₀, R₂₁, R₂₂, R₂₃, and R₂₃′ are each independently C1-C8 alkyl or C1-C8 substituted alkyl; or
further defined by the formula: 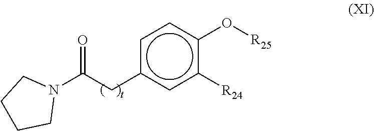
wherein:
t is 1, 2, or 3; and
R₂₄ and R₂₅ are each independently C1-C8 alkyl or C1-C8 substituted alkyl; or
further defined by the formula: 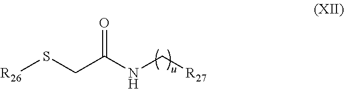
wherein:
u is 1, 2, 3, 4, or 5; and
R₂₆ and R₂₇ are each independently C6-C12 aryl, C6-C12 substituted aryl, C1-C12 heteroaryl or C1-C12 substituted heteroaryl; or
further defined by the formula: 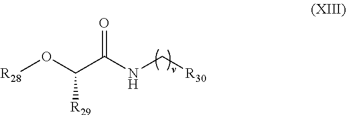
wherein:
v is 1, 2, 3, 4, or 5; and
R₂₈, R₂₉, and R₃₀ are each independently C1-C12 alkyl, C1-C12 substituted alkyl, C6-C12 aryl, C6-C12 substituted aryl, C1-C12 heteroaryl or C1-C12 substituted heteroaryl; and/or
(L) the compound is defined as shown in Table 1.

33. The method of claim 32, wherein the disease or disorder is associated with increased reactive oxygen species production.

34. The method of claim 32, wherein the disease or disorder is associated with decreased reactive oxygen species production.

35. The method of claim 32, wherein the disease or disorder is a disease or disorder may be treated with a change in reactive oxygen species.

36. The method of claim 35, wherein the change in reactive oxygen species is an increase in reactive oxygen species.

37. The method of claim 35, wherein the change in reactive oxygen species is a decrease in reactive oxygen species.

38.-72. (canceled)
